(19) United States
(12) Patent Application Publication
(10) Pub. No.: US 2009/0215878 A1
(43) Pub. Date: Aug. 27, 2009

(54) TREATMENT OF CHRONIC PAIN WITH ZINC FINGER PROTEINS

(75) Inventors: SIYUAN TAN, ALAMEDA, CA (US); STEVE H. ZHANG, RICHMOND, CA (US); JEFFREY C. MILLER, SAN LEANDRO, CA (US); PHILIP D. GREGORY, ORINDA, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SANGAMO BIOSCIENCES, INC., RICHMOND, CA (US)

(21) Appl. No.: 12/366,927

(22) Filed: Feb. 6, 2009

Related U.S. Application Data
(60) Provisional application No. 61/027,318, filed on Feb. 8, 2008.

(51) Int. Cl. A61K 31/7088; C12N 15/11; C07K 7/00

(52) U.S. Cl. 514/44 R; 536/23.1; 530/329

(57) ABSTRACT

A variety of zinc finger proteins (ZFPs) and methods utilizing such proteins are provided for use in treating chronic pain. ZFPs that bind to a target site in genes that are aberrantly expressed in subjects having chronic pain are described. In addition, ZFPs that bind to a target site in genes expressed at normal levels in subjects experiencing chronic pain, modulation of whose expression results in decreased pain perception, are also provided. For example, genes that are over-expressed in the dorsal root ganglia (DRG) of pain patients (e.g., Nav1.8) can be repressed.

### CROSS-REFERENCE TO RELATED APPLICATIONS

This application claims the benefit under 35 U.S.C. 119(e) of U.S. Provisional Patent Application No. 61/027,318, filed Feb. 8, 2008, which is incorporated herein by reference in its entirety for all purposes.

### REFERENCE TO A SEQUENCE LISTING

This application includes a sequence listing as shown in pages 1-16 of the Sequence Listing appended hereto.

### FIELD OF THE INVENTION

The invention resides in the fields of molecular genetics and medicine.

### BACKGROUND OF THE INVENTION

Chronic pain represents a variety of complex disorders that have diverse underlying pathology. Neuropathic pain, for example, often occurs as a result of injuries to the nerve, spinal cord or brain. There is evidence that nerve fibers in subjects with neuropathic pain develop abnormal excitability, particularly hyper-excitability. Zimmerman (2001) Eur J Pharmacol 429(1-3):23-37. Chronic inflammatory pain, on the other hand, is often a result of persistent tissue damage, which induces the release of neurotransmitters that mediate pain signaling. Although the American Pain Society estimates that nearly 50 million Americans are totally or partially disabled by pain, there are currently very few effective, well-tolerated treatments available. Wetzel et al. (1997) Ann Pharmacother 31(9):1082-3). Indeed, existing therapeutics cause a range of undesirable side effects primarily due to the difficulty in developing small-molecule drugs capable of specifically targeting the receptor/channel of choice.

Studies have shown the existence of primary sensory neurons that can be excited by noxious heat, mechanical damage, intense pressure or irritant chemicals, but not by innocuous stimuli such as warmth or light touch. These nociceptors selectively detect pain-inducing stimuli and appear to be distinct from other sensory mechanisms. This suggests that by suppressing the molecular mechanism of nociception it might be possible to limit the perception of painful stimuli without compromising general sensory awareness.

Transduction of noxious stimuli in nociception is mediated by cellular receptors that typically include neuron-specific sodium ion channels such as Nav1.8 (also referred to as PN3 or SCN10A). Nav1.8 is primarily expressed in neuronal cells of the peripheral sensory nervous system that are involved in both chronic and acute nociception, making them possible targets for therapeutic intervention aimed at limiting the pain response. Conventional therapeutic approaches typically focus on attempting to identify ligands that function as antagonists for these receptors. However, a major barrier to this approach is the cross-reactivity of receptor antagonists with other receptors of similar structure that are distinct from the pain-related targets.

The study of the molecular mechanisms triggering neuropathic pain has identified several genes that are abnormally expressed in sensory neurons of the Dorsal Root Ganglion (DRG) in models of neuropathic pain, including the sodium ion channel Nav1.8 (Coward et al. (2000) Pain 85(1-2):41-50). Reduction of Nav1.8 expression level has been shown to correlate with inhibition of neuropathic pain in the rat spinal nerve injury model of chronic pain (Lai et al. (2002) Pain 95(1-2):143-152).

The ability to alter expression of genes that encode pain signaling molecules (e.g. Nav1.8) may have utility in treating and/or preventing many forms of pain, including, but not limited to, neuropathic pain, inflammatory pain, cancer pain, thermal pain and mechanical pain.

U.S. provisional applications 60/560,535 (filed Apr. 8, 2004) and 60/576,757 (filed Jun. 2, 2004), as well as U.S. non-provisional application Ser. Nos. 11/096,706 (filed Apr. 1, 2005), 11/101,906 (filed Apr. 8, 2005) and 11/825,655 (filed Jul. 5, 2007) are directed to related subject matter and are incorporated by reference in their entireties for all purposes.

### BRIEF SUMMARY OF THE INVENTION

A variety of zinc finger proteins (ZFPs) and methods utilizing such proteins are provided for use in treating chronic pain. ZFPs that bind to a target site in Nav1.8, which is aberrantly expressed in subjects having chronic pain, are described.

The ZFPs can be fused to a regulatory domain as part of a fusion protein. By selecting a repression domain for fusion with the ZFP, one can repress gene expression and modulate physiological processes correlated with neuropathic pain.

In one aspect, the present invention includes a method for treating chronic pain in a subject by introducing a nucleic acid encoding a polypeptide comprising a transcriptional repression domain and a zinc finger DNA binding domain into the subject to repress transcription of the Nav1.8 gene. The encoded polypeptide is engineered, through manipulation of the nucleic acid sequence encoding the protein, to bind to a target site in Nav1.8 through the zinc finger DNA binding domain, and to repress transcription of the Nav1.8 gene through the transcriptional repression domain. In one embodiment, the transcriptional repression domain is a KRAB domain.

In another aspect, the present invention provides ZFPs, useful, for example, in the method for treating chronic pain, which comprise a zinc finger DNA binding domain including six zinc fingers denoted F1 through F6 in order from N-terminus to C-terminus. Each of the zinc fingers F1-F6 comprises a recognition region including seven amino acid residues capable of binding to a nucleic acid sequence of 3 nucleotides. The recognition regions of the six zinc fingers include four defined amino acid sequences, and two variable amino acid sequences. Zinc fingers F1, F3, F5 and F6 comprise recognition regions with a defined amino acid sequence as follows: F1=RSDVLSQ (SEQ ID NO:15); F3=RSDNLSR (SEQ ID NO:13); F5=QSGNLAR (SEQ ID NO:11); and F6=QSGNLAR (SEQ ID NO:11). Zinc fingers F2 and F4 comprise recognition regions with a variable amino acid sequence, as shown, for example, in Table 1, in which the DNA binding domain defined by the recognition regions of zinc fingers F1-F6 is capable of binding to a target site in the Nav1.8 gene comprising a nucleic acid sequence of 5′-GAAGAAgAATGAGAAGATG (SEQ ID NO:2). The ZFPs can further comprise a transcriptional repression domain such that binding of the ZFP to the Nav1.8 gene through the DNA binding domain represses transcription of Nav1.8. Alternatively, the ZFPs can further comprise a transcriptional activation domain such that binding of the ZFP to the Nav1.8 gene through the DNA binding domain activates transcription of Nav1.8.

Also provided herein are polynucleotides and nucleic acids that encode the ZFPs disclosed herein. Additionally, compositions containing the nucleic acids and/or ZFPs are also provided. For example, certain compositions include a nucleic acid that encodes one of the ZFPs described herein operably linked to a regulatory sequence, combined with a pharmaceutically acceptable carrier or diluent, wherein the regulatory sequence allows for expression of the nucleic acid in a cell. Protein-based compositions include a ZFP as disclosed herein and a pharmaceutically acceptable carrier or diluent.

The present invention also provides transgenic animals and recombinant cells comprising a transgene or polynucleotide molecule encoding the ZFPs disclosed herein. In some embodiments, the transgenic animal is a mammal (e.g., a rodent). The transgenes or polynucleotide molecules can include a transcriptional repression domain or a transcriptional activation domain in various embodiments. In some embodiments, the recombinant cells are mammalian cells (e.g., rat or mouse cells). In some cases, the recombinant cells are human cells.

The present invention also provides a method of screening compounds for activity to inhibit Nav1.8 gene expression or gene product activity. In some embodiments, the method includes providing a population of cells which express a transgene encoding a polypeptide having a zinc finger DNA-binding domain, as disclosed herein, in conjunction with a transcriptional activation domain, whereby expression of the transgene results in activation of Nav1.8 gene expression. In other embodiments, the cells express a REST-p65 construct, as described herein. The population of cells is contacted with a test compound, and a change in the expression or activity of a protein or mRNA is detected in the cell population to determine whether the test compound has activity to inhibit Nav1.8 gene expression or Nav1.8 gene product activity. In some embodiments, the change in expression or activity is detected in response to the test compound relative to the expression or activity of the protein or mRNA in a control population of cells that have not been contacted with the test compound. A decrease in the expression or activity is indicative of an inhibitory activity.

These and other embodiments will be readily apparent to those of ordinary skill in the art in view of the disclosure herein.

### BRIEF DESCRIPTION OF THE DRAWINGS

FIG. 1 is a graph depicting the increased (˜25 fold) expression of Nav1.8, normalized to human glyceraldehyde-3-phosphate dehydrogenase (GAPDH) mRNA, in human DAOY cells transduced with a lentiviral vector encoding REST-p65 (a fusion protein made by linking the DNA-binding domain from the RE1-silencing transcription factor to the transcription activation domain p65), as compared to untranduced (uninfected) and Green Fluorescent Protein (GFP) controls. The Nav1.8 gene is expressed almost exclusively in the peripheral sensory nervous system (dorsal root ganglia neurons and the sciatic nerve), and the increased Nav1.8 expression depicted in the graph illustrates the characteristics of a cell culture model designed for testing ZFP repressors of Nav1.8 that is representative of sensory neurons, which express high levels of Nav1.8.

FIG. 2 is a graph depicting repression of Nav1.8 gene expression, normalized to human GAPDH mRNA, in human DAOY cells transduced with a lentiviral vector encoding ZFP-TF 8982-KRAB. The DAOY cells were first transduced with a lentiviral vector encoding REST-p65 (a fusion protein made by linking the DNA-binding domain from the RE1-silencing transcription factor to the transcription activation domain p65) to elevate the basal level of Nav1.8 gene expression. Following stabilization of Nav1.8 mRNA at the elevated level (3-7 days), the cells were transduced with a lentiviral vector encoding a fusion of a KRAB A/B repression domain from KOX1 and a Nav1.8-targeted ZFP (designated 8982). The fusion protein is designated 8982-KRAB. “GFP” refers to a Green Fluorescent Protein control, and “mock” refers to a transduction absent a lentiviral vector.

FIG. 3 is a graph depicting repression of Nav1.8 gene expression, normalized to either rat GAPDH mRNA or rat peripherin (Prph) mRNA (a specific marker for sensory neurons), in rat dorsal root ganglion neurons transduced with a lentiviral vector encoding a fusion of a KRAB A/B repression domain from KOX1 and a Nav1.8-targeted ZFP (designated 8982). The fusion protein is designated 8982-KRAB. “GFP” refers to a Green Fluorescent Protein control.

FIG. 4 is a fluorescence micrograph showing a reduction of Nav1.8 protein levels in adult rat dorsal root ganglion sensory neurons transduced with a lentiviral vector encoding a fusion of a KRAB A/B repression domain from KOX1 and a Nav1.8-targeted ZFP (designated 8982). The fusion protein is designated 8982-KRAB. “Control ZFP TF” refers to a fusion of a KRAB A/B repression domain from KOX1 and an unrelated ZFP. Expression of Nav1.8 protein was visualized by immunostain of rabbit anti-Nav1.8 antibody (contacted with a green fluorescent anti-rabbit IgG), while the expression of ZFP TF was visualized by an immunostain of mouse anti-flag M2 monoclonal antibody (contacted with a red fluorescent anti-mouse IgG) (a flag epitope tag comprises a component of the ZFP constructs).

FIG. 5 is a graph depicting repression of Nav1.8 gene expression, normalized to rat GAPDH mRNA, in rat dorsal root ganglion neurons transduced with a herpes simplex virus (HSV) vector encoding a fusion of a KRAB A/B repression domain from KOX1 and a Nav1.8-targeted ZFP (designated 8982). The fusion protein is designated 8982-KRAB. “GFP” refers to a Green Fluorescent Protein control, and “mock” refers to a transduction absent an HSV vector.

FIG. 6 is a graph depicting the mechanical threshold, measured via an electronic Von Frey instrument, in rats induced to develop mechanical allodynia on the ipsilateral paw via ligation of the L5 spinal nerve (SNL rats). “HSV-8982” refers to SNL rats that were inoculated approximately 4 weeks after ligation with a herpes simplex virus (HSV) vector encoding a fusion of a KRAB A/B repression domain from KOX1 and a Nav1.8-targeted ZFP (designated 8982). The fusion protein is designated 8982-KRAB. “GFP” refers to SNL rats inoculated with an HSV vector containing a Green Fluorescent Protein control, and “No Vector” refers to SNL rats that received no inoculation. A range of control values taken from normal rats is represented by the bar at the top of the graph.

FIG. 7 is a graph depicting the functional activity of tetrodotoxin-resistant sodium channels, measured by recording the current in cultured rat neonatal dorsal root ganglia neurons via a whole-cell patch clamp, in the presence of a zinc finger fusion protein or a control. “8982-KRAB” refers to the transduced fusion protein comprising a fusion of a KRAB A/B repression domain from KOX1 and a Nav1.8-targeted ZFP.

### DETAILED DESCRIPTION OF THE INVENTION

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### I. DEFINITIONS

The term “zinc finger protein” or “ZFP” refers to a protein having DNA binding domains that are stabilized by zinc. The individual DNA binding domains are typically referred to as “fingers.” A ZFP has at least one finger, typically two, three, four, five, six or more fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. An exemplary motif characterizing one class of these proteins (C₂H₂ class) is -Cys-(X)₂₋₄-Cys-(X)₁₂-His-(X)₃₋₅-His (where X is any amino acid) (SEQ ID NO:1). Additional classes of zinc finger proteins are known and are useful in the practice of the methods, and in the manufacture and use of the compositions disclosed herein (see, e.g., Rhodes et al. (1993) Scientific American 268:56-65 and US Patent Application Publication No. 2003/0108880). Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

A “target site” is the nucleic acid sequence recognized by a ZFP. A single target site typically has about four to about ten base pairs. Typically, a two-fingered ZFP recognizes a four to seven base pair target site, a three-fingered ZFP recognizes a six to ten base pair target site, a four-finger ZFP recognizes a twelve to fourteen base pair target site and a six-fingered ZFP recognizes an eighteen to twenty base pair target site, which can comprise two adjacent nine to ten base pair target sites or three adjacent six to seven base pair target sites.

A “target subsite” or “subsite” is the portion of a DNA target site that is bound by a single zinc finger, excluding cross-strand interactions. Thus, in the absence of cross-strand interactions, a subsite is generally three nucleotides in length. In cases in which a cross-strand interaction occurs (i.e., a “D-able subsite,” see co-owned WO 00/42219) a subsite is four nucleotides in length and overlaps with another 3- or 4-nucleotide subsite.

“Kd” refers to the dissociation constant for a binding molecule, i.e., the concentration of a compound (e.g., a zinc finger protein) that gives half maximal binding of the compound to its target (i.e., half of the compound molecules are bound to the target) under given conditions (i.e., when [target]<<Kd), as measured using a given assay system (see, e.g., U.S. Pat. No. 5,789,538). The assay system used to measure the Kd should be chosen so that it gives the most accurate measure of the actual Kd of the ZFP. Any assay system can be used, as long is it gives an accurate measurement of the actual Kd of the ZFP. In one embodiment, the Kd for a ZFP is measured using an electrophoretic mobility shift assay (“EMSA”). Unless an adjustment is made for ZFP purity or activity, the Kd calculations may result in an overestimate of the true Kd of a given ZFP. Preferably, the Kd of a ZFP used to modulate transcription of a gene is less than about 100 nM, more preferably less than about 75 nM, more preferably less than about 50 nM, and most preferably less than about 25 nM.

“Chronic pain,” as used herein, means pain that is marked by a duration and/or frequency of recurrence that excludes acute pain of only limited duration and without recurrence. In some cases, “chronic pain” persists for a duration of six months or more, or longer than the temporal course of natural healing processes that may otherwise be associated with a particular injury, condition or disease. “Chronic pain” includes, without limitation, neuropathic pain, inflammatory pain, cancer pain, thermal pain and mechanical pain, or a combination of two or more of the foregoing.

A “gene,” for the purposes of the present disclosure, includes a DNA region encoding a gene product, as well as all DNA regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Nav1.8 is a gene involved in neuropathic pain.

Furthermore, the term “gene” includes nucleic acids that are substantially identical to a native gene. The terms “identical” or percent “identity,” in the context of two or more nucleic acids or polypeptides, refer to two or more sequences or subsequences that are the same or have a specified percentage of nucleotides or amino acid residues that are the same, when compared and aligned for maximum correspondence, as measured using a sequence comparison algorithm such as those described below, for example, or by visual inspection.

The term “gene product,” as used herein with reference to a particular gene includes RNA transcripts or polypeptides encoded thereby in any stage of generation, activation, inactivation, or degradation.

The phrase “substantially identical,” in the context of two nucleic acids or polypeptides, refers to two or more sequences or subsequences that have at least 75%, preferably at least 85%, more preferably at least 90%, 95% or higher or any integral value therebetween, nucleotide or amino acid residue identity, when compared and aligned for maximum correspondence, as measured using a sequence comparison algorithm such as those described below, for example, or by visual inspection. Preferably, the substantial identity exists over a region of the sequences that is at least about 10, preferably about 20, more preferably about 40-60 residues in length or any integral value therebetween, preferably over a longer region than 60-80 residues, more preferably at least about 90-100 residues, and most preferably the sequences are substantially identical over the full length of the sequences being compared, such as the coding region of a nucleotide sequence, for example.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection [see generally, Current Protocols in Molecular Biology, (Ausubel, F. M. et al., eds.) John Wiley & Sons, Inc., New York (1987-1999, including supplements such as supplement 46 (April 1999)]. Use of these programs to conduct sequence comparisons are typically conducted using the default parameters specific for each program.

Another example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. This is referred to as the neighborhood word score threshold (Altschul et al, supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For determining sequence similarity the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)). In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical is that the two molecules hybridize to each other under stringent conditions. “Hybridizes substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to”, refers to the binding, duplexing, or hybridizing of a molecule only to a particular nucleotide sequence under stringent conditions when that sequence is present in a complex mixture (e.g., total cellular DNA or RNA).

A further indication that two nucleic acid sequences or polypeptides are substantially identical is that the polypeptide encoded by the first nucleic acid is immunologically cross reactive with the polypeptide encoded by the second nucleic acid, as described below.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein that encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid that encodes a polypeptide is implicit in each described sequence.

A polypeptide is typically substantially identical to a second polypeptide, for example, where the two peptides differ only by conservative substitutions. A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. See, e.g., Creighton (1984) Proteins, W. H. Freeman and Company. In addition, individual substitutions, deletions or additions which alter, add or delete a single amino acid or a small percentage of amino acids in an encoded sequence are also “conservatively modified variations.”

A “functional fragment” or “functional equivalent” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid, binding to a regulatory molecule) are well known in the art. Similarly, methods for determining protein function are well known. For example, the DNA-binding function of a polypeptide can be determined by filter-binding, electrophoretic mobility-shift, or immunoprecipitation assays. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

The terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T. Thus, the term polynucleotide sequence is the alphabetical representation of a polynucleotide molecule. This alphabetical representation can be input into databases in a computer having a central processing unit and used for bioinformatics applications such as functional genomics and homology searching. The terms additionally encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs). The nucleotide sequences are displayed herein in the conventional 5′-3′ orientation.

“Chromatin” is the nucleoprotein structure comprising the cellular genome. “Cellular chromatin” comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4, and linker DNA (of variable length depending on the organism) extending between nucleosome cores. A molecule of histone HI is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

An “exogenous molecule” is a molecule that is not normally present in a cell, but can be introduced into a cell by one or more genetic, biochemical or other methods. Normal presence in the cell is determined with respect to the particular developmental stage and environmental conditions of the cell. Thus, for example, a molecule that is present only during embryonic development of muscle is an exogenous molecule with respect to an adult muscle cell. An exogenous molecule can comprise, for example, a functioning version of a malfunctioning endogenous molecule or a malfunctioning version of a normally functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., protein or nucleic acid (i.e., an exogenous gene), providing it has a sequence that is different from an endogenous molecule. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous molecule” is one that is normally present in a particular cell at a particular developmental stage under particular environmental conditions.

An “endogenous gene” is a gene that is present in its normal genomic and chromatin context. An endogenous gene can be present, e.g., in a chromosome, an episome, a bacterial genome or a viral genome.

The phrase “adjacent to a transcription initiation site” refers to a target site that is within about 50 bases either upstream or downstream of a transcription initiation site. “Upstream” of a transcription initiation site refers to a target site that is more than about 50 bases 5′ of the transcription initiation site (i.e., in the non-transcribed region of the gene). “Downstream” of a transcription initiation site refers to a target site that is more than about 50 bases 3′ of the transcription initiation site.

A “fusion molecule” is a molecule in which two or more subunit molecules are linked, typically covalently. The subunit molecules can be the same chemical type of molecule, or can be different chemical types of molecules. Examples of the first type of fusion molecule include, but are not limited to, fusion polypeptides (for example, a fusion between a ZFP DNA-binding domain and a transcriptional repression domain) and fusion nucleic acids (for example, a nucleic acid encoding the fusion polypeptide described supra). Examples of the second type of fusion molecule include, but are not limited to, a fusion between a triplex-forming nucleic acid and a polypeptide, and a fusion between a minor groove binder and a nucleic acid.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs that are modified, by processes such as capping, polyadenylation, methylation, and editing, and proteins modified by, for example, methylation, acetylation, phosphorylation, ubiquitination, ADP-ribosylation, myristilation, and glycosylation.

“Gene activation” refers to any process that results in an increase in production of a gene product. A gene product can be either RNA (including, but not limited to, mRNA, rRNA, tRNA, and structural RNA) or protein. Accordingly, gene activation includes those processes that increase transcription of a gene and/or translation of a mRNA. Examples of gene activation processes that increase transcription include, but are not limited to, those that facilitate formation of a transcription initiation complex, those that increase transcription initiation rate, those that increase transcription elongation rate, those that increase processivity of transcription and those that relieve transcriptional repression (by, for example, blocking the binding of a transcriptional repressor). Gene activation can constitute, for example, inhibition of repression as well as stimulation of expression above an existing level. Examples of gene activation processes that increase translation include those that increase translational initiation, those that increase translational elongation and those that increase mRNA stability. In general, gene activation comprises any detectable increase in the production of a gene product, in some instances an increase in production of a gene product by about 2-fold, in other instances from about 2- to about 5-fold or any integer therebetween, in still other instances between about 5- and about 10-fold or any integer therebetween, in yet other instances between about 10- and about 20-fold or any integer therebetween, sometimes between about 20- and about 50-fold or any integer therebetween, in other instances between about 50- and about 100-fold or any integer therebetween, and in yet other instances between 100-fold or more.

“Gene repression” and “inhibition of gene expression” refer to any process that results in a decrease in production of a gene product. A gene product can be either RNA (including, but not limited to, mRNA, rRNA, tRNA, and structural RNA) or protein. Accordingly, gene repression includes those processes that decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes that decrease translation include those that decrease translational initiation, those that decrease translational elongation and those that decrease mRNA stability. Transcriptional repression includes both reversible and irreversible inactivation of gene transcription. In general, gene repression comprises any detectable decrease in the production of a gene product, in some instances a decrease in production of a gene product by about 2-fold, in other instances from about 2- to about 5-fold or any integer therebetween, in yet other instances between about 5- and about 10-fold or any integer therebetween, in still other instances between about 10- and about 20-fold or any integer therebetween, sometimes between about 20- and about 50-fold or any integer therebetween, in other instances between about 50- and about 100-fold or any integer therebetween, and in still other instances 100-fold or more. In yet other instances, gene repression results in complete inhibition of gene expression, such that no gene product is detectable.

“Modulation” refers to a change in the level or magnitude of an activity or process. The change can be either an increase or a decrease. For example, modulation of gene expression includes both gene activation and gene repression. Modulation can be assayed by determining any parameter that is indirectly or directly affected by the expression of the target gene (e.g. Nav1.8). Such parameters include, e.g., changes in RNA or protein levels, changes in protein activity, changes in product levels, changes in downstream gene expression, changes in reporter gene transcription (luciferase, CAT, β-galactosidase, β-glucuronidase, green fluorescent protein (see, e.g., Mistili & Spector, Nature Biotechnology 15:961-964 (1997)); changes in signal transduction, phosphorylation and dephosphorylation, receptor-ligand interactions, second messenger concentrations (e.g., cGMP, cAMP, IP3, and Ca²⁺), cell growth, and vascularization. These assays can be in vitro, in vivo, and ex vivo. Such functional effects can be measured by any means known to those skilled in the art, e.g., measurement of RNA or protein levels, measurement of RNA stability, identification of downstream or reporter gene expression, e.g., via chemiluminescence, fluorescence, colorimetric reactions, antibody binding, inducible markers, ligand binding assays; changes in intracellular second messengers such as cGMP and inositol triphosphate (IP3); changes in intracellular calcium levels; cytokine release, and the like.

A “regulatory domain” or “functional domain” refers to a protein or a protein domain that has transcriptional modulation activity when tethered to a DNA binding domain, i.e., a ZFP. Typically, a regulatory domain is covalently or non-covalently linked to a ZFP (e.g., to form a fusion molecule) to effect transcription modulation. Regulatory domains can be activation domains or repression domains. Activation domains include, but are not limited to, VP16, VP64 and the p65 subunit of nuclear factor Kappa-B. Repression domains include, but are not limited to, KRAB, KOX, TIEG, MBD2B and v-ErbA. Additional regulatory domains include, e.g., transcription factors and co-factors (e.g., MAD, ERD, SID, early growth response factor 1, and nuclear hormone receptors), endonucleases, integrases, recombinases, methyltransferases, histone acetyltransferases, histone deacetylases etc. Activators and repressors include co-activators and co-repressors (see, e.g., Utley et al., Nature 394:498-502 (1998)). Alternatively, a ZFP can act alone, without a regulatory domain, to effect transcription modulation.

The term “operably linked” or “operatively linked” is used with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. An operatively linked transcriptional regulatory sequence is generally joined in cis with a coding sequence, but need not be directly adjacent to it. For example, an enhancer can constitute a transcriptional regulatory sequence that is operatively linked to a coding sequence, even though they are not contiguous.

With respect to fusion polypeptides, the term “operably linked” or “operatively linked” can refer to the fact that each of the components performs the same function in linkage to the other component as it would if it were not so linked. For example, with respect to a fusion polypeptide in which a ZFP DNA-binding domain is fused to a transcriptional repression domain (or functional fragment thereof), the ZFP DNA-binding domain and the transcriptional repression domain (or functional fragment thereof) are in operative linkage if, in the fusion polypeptide, the ZFP DNA-binding domain portion is able to bind its target site and/or its binding site, while the transcriptional repression domain (or functional fragment thereof) is able to repress transcription.

The term “recombinant,” when used with reference to a cell, indicates that the cell replicates an exogenous nucleic acid, or expresses a peptide or protein encoded by an exogenous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette,” “expression cassette” or “expression construct” is a nucleic acid construct, generated recombinantly or synthetically, that has control elements that are capable of effecting expression of a structural gene that is operatively linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding a desired polypeptide) and a promoter. Additional factors necessary or helpful in effecting expression can also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

A “promoter” is defined as an array of nucleic acid control sequences that direct transcription. As used herein, a promoter typically includes necessary nucleic acid sequences near the start site of transcription, such as, in the case of certain RNA polymerase II type promoters, a TATA element, CCAAT box, SP-1 site, etc. As used herein, a promoter also optionally includes distal enhancer or repressor elements, which can be located as much as several thousand base pairs from the start site of transcription. The promoters often have an element that is responsive to transactivation by a DNA-binding moiety such as a polypeptide, e.g., a nuclear receptor, Gal4, the lac repressor and the like.

A “constitutive” promoter is a promoter that is active under most environmental and developmental conditions. An “inducible” promoter is a promoter that is active under certain environmental or developmental conditions.

A “weak promoter” refers to a promoter having about the same activity as a wild type herpes simplex virus (“HSV”) thymidine kinase (“tk”) promoter or a mutated HSV tk promoter, as described in Eisenberg & McKnight, Mol. Cell. Biol. 5:1940-1947 (1985).

An “expression vector” is a nucleic acid construct, generated recombinantly or synthetically, with a series of specified nucleic acid elements that permit transcription of a particular nucleic acid in a host cell, and optionally integration or replication of the expression vector in a host cell. The expression vector can be part of a plasmid, virus, or nucleic acid fragment, of viral or non-viral origin. Typically, the expression vector includes an “expression cassette,” which comprises a nucleic acid to be transcribed operably linked to a promoter. The term expression vector also encompasses naked DNA operably linked to a promoter.

By “host cell” is meant a cell that contains an expression vector or nucleic acid, either of which optionally encodes a ZFP or a ZFP fusion protein. The host cell typically supports the replication or expression of the expression vector. Host cells can be prokaryotic cells such as, for example, E. coli, or eukaryotic cells such as yeast, fungal, protozoal, higher plant, insect, or amphibian cells, or mammalian cells such as CHO, HeLa, 293, COS-1, and the like, e.g., cultured cells (in vitro), explants and primary cultures (in vitro and ex vivo), and cells in vivo.

The term “naturally occurring,” as applied to an object, means that the object can be found in nature, as distinct from being artificially produced by humans.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The polypeptide sequences are displayed herein in the conventional N-terminal to C-terminal orientation.

A “subsequence” or “segment” when used in reference to a nucleic acid or polypeptide refers to a sequence of nucleotides or amino acids that comprise a part of a longer sequence of nucleotides or amino acids (e.g., a polypeptide), respectively.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage.

By an “effective” amount (or “therapeutically effective” amount) of a pharmaceutical composition is meant a sufficient, but nontoxic amount of the agent to provide the desired effect. The term refers to an amount sufficient to treat a subject. Thus, the term therapeutic amount refers to an amount sufficient to remedy a disease state or symptoms, by preventing, hindering, retarding or reversing the progression of the disease or any other undesirable symptoms whatsoever. The term prophylactically effective amount refers to an amount given to a subject that does not yet have the disease, and thus is an amount effective to prevent, hinder or retard the onset of a disease.

### II. OVERVIEW

A variety of compositions and methods are provided herein for modulating the expression of a target gene that is often over-expressed in subjects with chronic pain. The ability to alter expression of genes that encode pain signaling molecules (e.g., Nav1.8) is useful for treating and/or preventing many forms of pain, including without limitation, neuropathic pain, inflammatory pain, cancer pain, thermal pain and mechanical pain. In some individuals, pain may result from one, or two or more of the foregoing.

Zinc finger proteins (ZFPs) that are capable of repressing expression of Nav1.8, a gene involved in nerve excitability, are described herein. The ZFPs comprise a DNA-binding domain that is engineered to specifically recognize and bind to a particular nucleic acid segment (target site) in Nav1.8. In one embodiment, the ZFPs are linked to regulatory domains to create chimeric transcription factors to repress Nav1.8 gene expression. Also described are methods for treating chronic pain by contacting a cell or population of cells, such as in an organism, with one or more ZFPs that bind to a specific target site within the Nav1.8 gene to thereby repress expression of the gene via an operatively linked transcriptional repression domain.

The tetrodotoxin-resistant sodium channel (Nav1.8, also known as PN3, SNS, and SCN10A) is restricted to the peripheral small diameter sensory neurons in the dorsal root ganglion (DRG) and is believed to play a unique role in transmission of nociceptive information to the spinal cord. Its expression is also influenced by nerve growth factor (NGF) and tyrosine Kinase Receptor A (TrkA). Nav1.8^{-/-} mice are apparently normal but show deficits in thermoreception and the development of inflammatory pain, and their behavioral responses to noxious mechanical stimulation appear to be completely abolished.

By virtue of the ability of the ZFPs to bind to a target site and influence expression of Nav1.8, the ZFPs provided herein can be used to ameliorate or eliminate neuropathic pain. In certain applications, the ZFPs can be used to repress expression of Nav1.8 in subjects with neuropathic pain, both in vitro and in vivo. Such repression can be utilized, for example, as treatment for chronic pain.

In addition, inactivation of genes involved in pain perception such as, for example, Nav1.8, can be used for treatment of neuropathic pain. In these embodiments, fusion proteins comprising an engineered zinc finger domain and a cleavage domain (or cleavage half-domain) are used for targeted cleavage of a DNA sequence in an endogenous gene involved in neuropathic pain. Targeted cleavage can result in the subsequent introduction of a mutation into the cleaved gene by non-homologous end-joining; alternatively, one or more sequences can be inserted into a gene by homologous recombination following targeted cleavage. See U.S. Patent Application Publication Nos. 2003/0232410; 2005/0026157; 2005/0064474 and WO 03/87341 for additional details relating to targeted cleavage and recombination.

Disclosed herein are compositions and methods for targeted regulation of transcription and targeted DNA cleavage, which are useful, for example, in the treatment of neuropathic pain. These include fusion proteins comprising an engineered zinc finger protein and a functional domain such as, for example, a transcriptional repression domain, a nuclease domain or a nuclease half-domain. Suitable functional domains are known in the art and include, without limitation, transcriptional repression domains such as, for example, KRAB A/B, KOX, TGF-beta-inducible early gene (TIEG) and v-erbA, cleavage domains such as, for example, HO and cleavage half-domains such as, for example, the cleavage domain of FokI. One or more of the same or different functional domains can be present in a given fusion protein. See co-owned U.S. Patent Application Publication No. 2002/0160940, incorporated by reference, for disclosure of exemplary transcriptional repression domains. Co-owned U.S. Patent Application Publication No. 2005/0064474, incorporated by reference, discloses exemplary cleavage domains and cleavage half-domains.

### III. ZINC FINGER PROTEINS FOR REGULATING GENE EXPRESSION

### A. General

The zinc finger proteins (ZFPs) disclosed herein are proteins that can bind to DNA in a sequence-specific manner. As indicated above, these ZFPs can be used to modulate expression of a target gene (e.g., a gene involved in nerve excitability) in vivo or in vitro and by so doing treat chronic pain. An exemplary motif characterizing one class of these proteins, the C₂H₂ class, is -Cys-(X)₂₋₄-Cys-(X)₁₂-His-(X)₃₋₅-His (where X is any amino acid) (SEQ ID NO:1). Several structural studies have demonstrated that the finger domain contains an alpha helix containing the two invariant histidine residues and two invariant cysteine residues in a beta turn coordinated through zinc. However, the ZFPs provided herein are not limited to this particular class. Additional classes of zinc finger proteins are known and can also be used in the methods and compositions disclosed herein. See, e.g., Rhodes, et al. (1993) Scientific American 268:56-65 and US Patent Application Publication No. 2003/0108880. In certain ZFPs, a single finger domain is about 30 amino acids in length. Zinc finger domains are involved not only in DNA-recognition, but also in RNA binding and in protein-protein binding.

The x-ray crystal structure of Zif268, a three-finger domain from a murine transcription factor, has been solved in complex with a cognate DNA-sequence and shows that each finger can be superimposed on the next by a periodic rotation. The structure suggests that each finger interacts independently with DNA over 3 base-pair intervals, with side-chains at positions −1, +2, +3 and +6 on each recognition helix making contacts with their respective DNA triplet subsites. Numbering is with respect to the beginning of the helical portion of the zinc finger; in this numbering scheme, the first (or amino terminal-most) conserved histidine residue of the zinc finger is designated +7. The amino terminus of Zif268 is situated at the 3′ end of the DNA strand with which it makes most contacts. Some zinc fingers can bind to a fourth base in a target segment. If the strand with which a zinc finger protein makes most contacts is designated the target strand, some zinc finger proteins bind to a three base triplet in the target strand and a fourth base on the nontarget strand. The fourth base is complementary to the base immediately 3′ of the three base subsite.

### B. Zinc Finger Proteins Targeted to the Nav1.8 Gene

The methods for pain therapy and analgesia disclosed herein involve regulation of the expression of, inter alia, the endogenous cellular gene encoding Nav1.8 (also known as PN3) by expressing, in one or more cells of a subject, a fusion protein that binds to a target sequence in the Nav1.8 gene and represses its transcription. Such a fusion protein can be expressed in a cell by introducing into the cell a nucleic acid (DNA or RNA) that encodes the protein, or by introducing the protein directly into the cell. Nucleic acids and/or proteins can also be administered to a subject (see below) such that the nucleic acid or protein enters one or more cells of the subject. In addition, nucleic acids and/or proteins can be introduced ex vivo into cells which have been isolated from a subject, said cells being returned to the subject after introduction of the nucleic acid and/or protein and optional incubation.

In certain embodiments, a fusion protein as described above comprises a DNA-binding domain and a functional domain (e.g., a transcriptional repression domain). The DNA-binding domain can be an engineered zinc finger binding domain as described, for example, in co-owned U.S. Pat. Nos. 6,453,242; 6,534,261; 6,607,882; 6,785,613; 6,794,136 and 6,824,978. See also, for example, U.S. Pat. Nos. 5,5,789,538; 6,007,988; 6,013,453; 6,140,466; 6,242,568; 6,410,248; 6,479,626; 6,746,838 and 6,790,941.

The DNA-binding domain can bind to any sequence, in the transcribed or non-transcribed region of the Nav1.8 gene, or to any other sequence, as long as transcription of the Nav1.8 gene is regulated. Methods for selecting target sites for binding by zinc finger proteins are disclosed in co-owned U.S. Pat. No. 6,453,242. In certain embodiments, the target site is in an accessible region of cellular chromatin as described, for example, in co-owned U.S. Patent Application Publication No. 2002/0064802 A1.

For those embodiments in which the DNA-binding domain is an engineered zinc finger binding domain, the zinc finger domain is engineered to bind a specific target site. The binding domain contains a plurality of zinc fingers (e.g., 2, 3, 4, 5, 6 or more zinc fingers). In general, an individual zinc finger binds a subsite of 3-4 nucleotides. The subsites can be contiguous in a target site (and are in some cases overlapping); alternatively a subsite can be separated from an adjacent subsite by gaps of one, two three or more nucleotides. Binding to subsites separated by a gap of one or more nucleotides is facilitated by the use of non-canonical, longer linker sequences between adjacent zinc fingers. See, for example, U.S. Pat. No. 6,479,626 and U.S. Patent Application Publication Nos. 2002/0173006 and 2003/0119023.

Human Nav1.8 expression is regulated by ZFPs through binding to a target site with the following nucleic acid sequence: 5′-GAAGAAgAATGAGAAGATG (SEQ ID NO:2) or a subsequence thereof. Rat Nav1.8 expression is regulated by the ZFPs through binding to a target site with the following nucleic acid sequence: 5′-CAAGAAgAATGAGAAGATG (SEQ ID NO:3). Species variants of NAV1.8 can be regulated at the corresponding site (i.e., site having greatest sequence identity) to SEQ ID NO:2 or 3 in that species. Nucleotides comprising subsites to which individual zinc fingers primarily contact are shown in uppercase. Nucleotides between subsites are shown in lowercase. The target site for the human Nav1.8 gene, identified above, is positioned such that the three nucleotides at the 3′ end of the target sequence overlap with the initiation codon for the Nav1.8 gene. This nucleotide, “A” in the sequence “ATG” (SEQ ID NO:10), is located at position 38810505 on the minus strand of human chromosome 3 (i.e., the “T” of the ATG (SEQ ID NO:10) codon is located at position 38810504). See Homo sapiens Genome (build 35.1), NCBI.

Exemplary zinc finger binding domains that bind to this target site are shown in Table 1 and in the Examples. Table 1 shows the amino acid sequence of the seven-residue recognition region of each zinc finger (amino acid residues −1 through +6 with respect to the start of the helical portion of the zinc finger), for each of the six fingers, denoted F1 through F6 in order from N-terminus to C-terminus.

TABLE 1

| | | Finger | | | | | |
|---|---|---|---|---|---|---|---|
| ZFP TF | | F6 | F5 | F4 | F3 | F2 | F1 |
| | Target | 5′-GAA | GAAg | AAT | GAG | AAG | ATG |
| | Triplet: | (SEQ ID NO: 4) | (SEQ ID NO: 6) | (SEQ ID NO: 7) | (SEQ ID NO: 8) | (SEQ ID NO: 9) | (SEQ ID NO: 10) |
| | | or CAA | | | | | |
| | | (SEQ ID NO: 5) | | | | | |
| 8982 | Design | QSGNLAR | QSGNLAR | TNQNRIT | RSDNLSR | RSDNLSV | RSDVLSQ |
| | | (SEQ ID NO: 11) | (SEQ ID NO: 11) | (SEQ ID NO: 12) | (SEQ ID NO: 13) | (SEQ ID NO: 14) | (SEQ ID NO: 15) |
| 11615 | Design | QSGNLAR | QSGNLAR | TNQNRIT | RSDNLSR | YSRGLWA | RSDVLSQ |
| | | (SEQ ID NO: 11) | (SEQ ID NO: 11) | (SEQ ID NO: 12) | (SEQ ID NO: 13) | (SEQ ID NO: 16) | (SEQ ID NO: 15) |
| 11618 | Design | QSGNLAR | QSGNLAR | TNQNRIT | RSDNLSR | WPGSLSN | RSDVLSQ |
| | | (SEQ ID NO: 11) | (SEQ ID NO: 11) | (SEQ ID NO: 12) | (SEQ ID NO: 13) | (SEQ ID NO: 17) | (SEQ ID NO: 15) |
| 11619 | Design | QSGNLAR | QSGNLAR | TNQNRIT | RSDNLSR | WRPNLVA | RSDVLSQ |
| | | (SEQ ID NO: 11) | (SEQ ID NO: 11) | (SEQ ID NO: 12) | (SEQ ID NO: 13) | (SEQ ID NO: 18) | (SEQ ID NO: 15) |
| 12640 | Design | QSGNLAR | QSGNLAR | TNQNRIT | RSDNLSR | APRYLWQ | RSDVLSQ |
| | | (SEQ ID NO: 11) | (SEQ ID NO: 11) | (SEQ ID NO: 12) | (SEQ ID NO: 13) | (SEQ ID NO: 19) | (SEQ ID NO: 15) |
| 12642 | Design | QSGNLAR | QSGNLAR | TNQNRIT | RSDNLSR | LLKYLAT | RSDVLSQ |
| | | (SEQ ID NO: 11) | (SEQ ID NO: 11) | (SEQ ID NO: 12) | (SEQ ID NO: 13) | (SEQ ID NO: 20) | (SEQ ID NO: 15) |
| 12668 | Design | QSGNLAR | QSGNLAR | LKRTLMV | RSDNLSR | APRYLWQ | RSDVLSQ |
| | | (SEQ ID NO: 11) | (SEQ ID NO: 11) | (SEQ ID NO: 21) | (SEQ ID NO: 13) | (SEQ ID NO: 19) | (SEQ ID NO: 15) |
| 12695 | Design | QSGNLAR | QSGNLAR | LLQTLSS | RSDNLSR | SSRYLWQ | RSDVLSQ |
| | | (SEQ ID NO: 11) | (SEQ ID NO: 11) | (SEQ ID NO: 22) | (SEQ ID NO: 13) | (SEQ ID NO: 23) | (SEQ ID NO: 15) |
| 12696 | Design | QSGNLAR | QSGNLAR | LLQTLSS | RSDNLSR | APRYLWQ | RSDVLSQ |
| | | (SEQ ID NO: 11) | (SEQ ID NO: 11) | (SEQ ID NO: 22) | (SEQ ID NO: 13) | (SEQ ID NO: 19) | (SEQ ID NO: 15) |
| 12697 | Design | QSGNLAR | QSGNLAR | LLQTLSS | RSDNLSR | HPRYLWQ | RSDVLSQ |
| | | (SEQ ID NO: 11) | (SEQ ID NO: 11) | (SEQ ID NO: 22) | (SEQ ID NO: 13) | (SEQ ID NO: 24) | (SEQ ID NO: 15) |
| 14332 | Design | QSGNLAR | QSGNLAR | LHRTLTV | RSDNLSR | QRRYLWA | RSDVLSQ |
| | | (SEQ ID NO: 11) | (SEQ ID NO: 11) | (SEQ ID NO: 25) | (SEQ ID NO: 13) | (SEQ ID NO: 26) | (SEQ ID NO: 15) |
| 14348 | Design | QSGNLAR | QSGNLAR | VRCNLTK | RSDNLSR | QKRYLWQ | RSDVLSQ |
| | | (SEQ ID NO: 11) | (SEQ ID NO: 11) | (SEQ ID NO: 27) | (SEQ ID NO: 13) | (SEQ ID NO: 28) | (SEQ ID NO: 15) |
| 14356 | Design | QSGNLAR | QSGNLAR | LRRTLHM | RSDNLSR | QKRYLWQ | RSDVLSQ |
| | | (SEQ ID NO: 11) | (SEQ ID NO: 11) | (SEQ ID NO: 29) | (SEQ ID NO: 13) | (SEQ ID NO: 28) | (SEQ ID NO: 15) |
| 14365 | Design | QSGNLAR | QSGNLAR | LKNALRI | RSDNLSR | QKRYLWQ | RSDVLSQ |
| | | (SEQ ID NO: 11) | (SEQ ID NO: 11) | (SEQ ID NO: 30) | (SEQ ID NO: 13) | (SEQ ID NO: 28) | (SEQ ID NO: 15) |

The amino acid residues shown in Table 1 correspond to residues −1 through +6 with respect to the start of the alpha-helical portion of a zinc finger and are denoted the “recognition regions” because one or more of these residues participate in sequence specificity of nucleic acid binding. Accordingly, proteins comprising the same recognition regions in any polypeptide backbone sequence are considered equivalents to the proteins identified in Table 1 since they have the same DNA-binding specificity. The residues shown for fingers F1, F3, F5 and F6 are the same in the above proteins, whereas the residues for fingers F2 and F4 vary among different proteins.

Thus, in certain embodiments, the recognition regions disclosed in Table 1 can be present in any zinc finger backbone (see, e.g., U.S. Pat. Nos. 6,453,242 and 6,534,261) and the resulting proteins can be used to regulate transcription, e.g., in the treatment of neuropathic pain.

Within the recognition region, residues −1, +3 and +6 are primarily responsible for protein-nucleotide contacts. The residue at position +2 is also sometimes involved in binding specificity. Accordingly, non-limiting examples of additional equivalents include zinc fingers containing residues at positions −1, +3 and +6 (and optionally +2) that are identical to those of any of the zinc fingers disclosed herein.

Correspondences between amino acids at the −1, +3 and +6 (and optionally +2) contact residues of the recognition region of a zinc finger, and nucleotides in a target site, have been described. See, for example, U.S. Pat. Nos. 6,007,988; 6,013,453; 6,746,838; and 6,866,997; as well as PCT Publications WO 96/06166; WO 98/53058; WO 98/53059 and WO 98/53060. Accordingly, also to be considered equivalents are zinc finger proteins having the same binding specificity, according to the aforementioned design rules, as the proteins disclosed herein.

### IV. CHARACTERISTICS OF ZFPS

Zinc finger proteins are formed from zinc finger components. For example, zinc finger proteins can have one to thirty-seven fingers, commonly having 2, 3, 4, 5 or 6 fingers. Preferably zinc finger proteins have six zinc fingers. A zinc finger protein recognizes and binds to a target site (sometimes referred to as a target segment) that represents a relatively small subsequence within a target gene. Each component finger of a zinc finger protein can bind to a subsite within the target site. The subsite includes a triplet of three contiguous bases all on the same strand (sometimes referred to as the target strand). The subsite may or may not also include a fourth base on the opposite strand that is the complement of the base immediately 3′ of the three contiguous bases on the target strand. As described previously, the present invention provides ZFPs designed to bind to a target site comprising a nucleic acid sequence of 5′-GAAGAAgAATGAGAAGATG (SEQ ID NO:2). In this particular instance, each of the six zinc fingers binds a triplet subsite within this target sequence, the lower case nucleotide representing an unbound “gap” between the two adjacent subsites. In many zinc finger proteins, a zinc finger binds to its triplet subsite substantially independently of other fingers in the same zinc finger protein. Accordingly, the binding specificity of a zinc finger protein containing multiple fingers is usually approximately the aggregate of the specificities of its component fingers. For example, the invention provides zinc finger proteins formed from first, second, third, fourth, fifth, and sixth fingers that individually bind to triplets AAA, BBB, CCC, DDD, EEE, and FFF, the binding specificity of the zinc finger protein is 3′AAA BBB CCC DDD EEE FFF5′.

The relative order of fingers in a zinc finger protein from N-terminal to C-terminal determines the relative order of triplets in the 3′ to 5′ direction in the target. For example, the invention provides ZFPs comprising six zinc fingers designated F1, F2, F3, F4, F5, and F6 in order from N-terminus to C-terminus. These zinc fingers are designed to bind to a target sequence of 5′-GAAGAAgAATGAGAAGATG (SEQ ID NO:2). Finger F1 binds the 3′ ATG (SEQ ID NO:10), finger F2 binds the upstream AAG (SEQ ID NO:9), finger F3 binds the upstream GAG (SEQ ID NO:8), finger F4 binds the upstream AAT (SEQ ID NO:7), finger F5 binds the upstream GAA (separated from the F4 subsite by the lower case g, which is not bound, and which, with GAA, corresponds to SEQ ID NO:6), and finger F6 binds the 5′ GAA (SEQ ID NO:4) subsite. Some of the fingers also have cross-strand interactions with a fourth base as described in WO 00/42219. The assessment of binding properties of a zinc finger protein as the aggregate of its component fingers may, in some cases, be influenced by context-dependent interactions of multiple fingers binding in the same protein.

The zinc fingers of the DNA binding domain of the above zinc finger proteins each comprise a recognition region including seven amino acid residues capable of binding to the corresponding nucleotide subsite discussed above. Examples of suitable proteins are shown in Table 1. A protein can be formed by combining the fingers F1, F2, F3, F4, F5 and F6 shown in the same row of the Table. Additional zinc finger proteins can be made by using fingers F1 (RSDVLSQ (SEQ ID NO:15)); F3=RSDNLSR (SEQ ID NO:13); F5=QSGNLAR (SEQ ID NO:11); and F6=QSGNLAR (SEQ ID NO:11). in Table 1 as a core, and combining with fingers F2 and F4 from different rows of the Table. Alternatively, the core sequences of F1, F3, F5 and F6 can be combined with other zinc fingers designed or selected as described below that bind to the same triplet target subsites as F2 and F4 in the Table.

Zinc finger proteins can also be constructed having fewer than six fingers to bind subsequences of the target site 5′ GAAGAAgAATGAGAAGATG3′ (SEQ ID NO:2) For example, a zinc finger protein including fingers F2, F3, F4 and F5 as described in Table 1 but lacking fingers F1 and F6 binds to the sequence GAAgAATGAGAAG (SEQ ID NO:60). A zinc finger protein including fingers F1, F3, F4, F5 and F6 as defined in Table 1 binds to the sequence GAAGAAgAATGAGaagATG (SEQ ID NO:2).

Two or more zinc finger proteins can be linked to have a target specificity that is the aggregate of that of the component zinc finger proteins (see e.g., Kim & Pabo, Proc. Natl. Acad. Sci. U.S.A. 95, 2812-2817 (1998)). For example, a first zinc finger protein having first, second and third component fingers that respectively bind to XXX, YYY and ZZZ can be linked to a second zinc finger protein having first, second and third component fingers with binding specificities, AAA, BBB and CCC. The binding specificity of the combined first and second proteins is thus 3′XXXYYYZZZ_AAABBBCCC5′, where the underline indicates a short intervening region (typically 0-5 bases of any type). In this situation, the target site can be viewed as comprising two target segments separated by an intervening segment.

Linkage can be accomplished using any of the following peptide linkers:

(SEQ ID NO:31)

T G E K P: (Liu et al., 1997, supra.);

(SEQ ID NO:32)

(G4S)n (Kim et al., Proc. Natl. Acad. Sci. U.S.A.
93:1156-1160 (1996.);

(SEQ ID NO:33)

GGRRGGGS;

(SEQ ID NO:34)

LRQRDGERP;

(SEQ ID NO:35)

LRQKDGGGSERP;

(SEQ ID NO:36)

LRQKD(G₃S)₂ERP.

Alternatively, flexible linkers can be rationally designed using computer programs capable of modeling both DNA-binding sites and the peptides themselves or by phage display methods. In a further variation, noncovalent linkage can be achieved by fusing two zinc finger proteins with domains promoting heterodimer formation of the two zinc finger proteins. For example, one zinc finger protein can be fused with fos and the other with jun (see Barbas et al., WO 95/119431).

Linkage of two zinc finger proteins is advantageous for conferring a unique binding specificity within a mammalian genome. A typical mammalian diploid genome consists of 3×10⁹ bp. Assuming that the four nucleotides A, C, G, and T are randomly distributed, a given 9 bp sequence is present approximately 23,000 times. Thus a ZFP recognizing a 9 bp target with absolute specificity would have the potential to bind to about 23,000 sites within the genome. An 18 bp sequence is present about once in a random DNA sequence whose complexity is ten times that of a mammalian genome.

A component finger of zinc finger protein typically contains about 30 amino acids and, in one embodiment, has the following motif (N—C):

(SEQ ID NO:37)

Cys-(X)₂₋₄-Cys-X.X.X.X.X.X.X.X.X.X.X.X-His-(X)₃₋₅-
His

The two invariant histidine residues and two invariant cysteine residues in a single beta turn are coordinated through a zinc atom (see, e.g., Berg & Shi, Science 271, 1081-1085 (1996)). The above motif shows a numbering convention that is standard in the field for the region of a zinc finger conferring binding specificity. The amino acid on the left (N-terminal side) of the first invariant His residue is assigned the number +6, and other amino acids further to the left are assigned successively decreasing numbers. The alpha helix begins at residue 1 and extends to the residue following the second conserved histidine. The entire helix is therefore of variable length, between 11 and 13 residues.

### V. DESIGN OF ZFPS

The recognition sequences for each zinc finger described above are combined with framework residues and individual fingers including the framework residues are linked to one another to form a zinc finger protein. A natural ZFP can provide a source of framework residues (i.e., residues other than at positions −1 to +6). One suitable ZFP is the DNA binding domain of the mouse transcription factor Zif268. The DNA binding domain of this protein has the amino acid sequence:

| | | |
|---|---|---|
| YACPVESCDRRFSRSDELTRHIRIHTGQKP | (F1) | (SEQ ID NO:38) |
| FQCRICMRNFSRSDHLTTHIRTHTGEKP | (F2) | (SEQ ID NO:39) |
| FACDICGRKFARSDERKRHTKILHLRQK | (F3) | SEQ ID NO:40) |
| and | | |
| binds to a target | | |
| 5′ GCG TGG GCG 3′. | | (SEQ ID NO:41) |

Another suitable natural zinc finger protein as a source of framework residues is Sp-1. The Sp-1 sequence used for construction of zinc finger proteins corresponds to amino acids 531 to 624 in the Sp-1 transcription factor. This sequence is 94 amino acids in length. See, e.g., U.S. Patent Application No. 20030021776 for the sequence of Sp1 and an alternate form of Sp-1, referred to as an Sp-1 consensus sequence.

Sp-1 binds to a target site 5′GGG GCG GGG3′ (SEQ ID NO:42).

If any additional zinc fingers are desired for combination with the core F1, F3, F5 and F6 sequences above, they can be designed in accordance with a number of substitution rules that assist rational design of some zinc finger proteins. For example, ZFP DNA-binding domains can be designed and/or selected to recognize a particular target site as described in U.S. Pat. Nos. 6,453,242; 6,534,261; 6,746,838; 6,785,613; 6,794,136; and 6,866,997; U.S. Patent Application Publication No. 2003/0104526; as well as U.S. Pat. Nos. 5,789,538; 6,007,408; 6,013,453; 6,140,081; and 6,140,466; and PCT publications WO 95/19431, WO 98/53058; WO 98/53059; WO 98/53060; WO 98/54311, WO 00/23464 and WO 00/27878. Alternatively, the technique of phage display provides a largely empirical means of generating zinc finger proteins with desired target specificity (see e.g., Rebar, U.S. Pat. No. 5,789,538; Choo et al., WO 96/06166; Barbas et al., WO 95/19431 and WO 98/543111.

### VI. PRODUCTION OF ZINC FINGER PROTEINS

### A. Synthesis and Cloning

ZFP polypeptides and nucleic acids encoding the same can be made using routine techniques in the field of recombinant genetics. Basic texts disclosing general methods include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)). In addition, nucleic acids less than about 100 bases can be custom ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.). Similarly, peptides can be custom ordered from any of a variety of sources, such as PeptidoGenic (pkim@ccnet.com), HTI Bio-products, Inc. (http://www.htibio.com), BMA Biomedicals Ltd (U.K.), Bio.Synthesis, Inc.

Oligonucleotides can be chemically synthesized according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides is by either denaturing polyacrylamide gel electrophoresis or by reverse phase HPLC. The sequence of the cloned genes and synthetic oligonucleotides can be verified after cloning using, e.g., the chain termination method for sequencing double-stranded templates of Wallace et al., Gene 16:21-26 (1981).

Two alternative methods are typically used to create the coding sequences required to express newly designed DNA-binding peptides. One protocol is a PCR-based assembly procedure that utilizes six overlapping oligonucleotides. Three oligonucleotides correspond to “universal” sequences that encode portions of the DNA-binding domain between the recognition helices. These oligonucleotides typically remain constant for all zinc finger constructs. The other three “specific” oligonucleotides are designed to encode the recognition helices. These oligonucleotides contain substitutions primarily at positions −1, 2, 3 and 6 on the recognition helices making them specific for each of the different DNA-binding domains.

The PCR synthesis is carried out in two steps. First, a double stranded DNA template is created by combining the six oligonucleotides (three universal, three specific) in a four cycle PCR reaction with a low temperature annealing step, thereby annealing the oligonucleotides to form a DNA “scaffold.” The gaps in the scaffold are filled in by high-fidelity thermostable polymerase, the combination of Taq and Pfu polymerases also suffices. In the second phase of construction, the zinc finger template is amplified by external primers designed to incorporate restriction sites at either end for cloning into a shuttle vector or directly into an expression vector.

An alternative method of cloning the newly designed DNA-binding proteins relies on annealing complementary oligonucleotides encoding the specific regions of the desired ZFP. This particular application requires that the oligonucleotides be phosphorylated prior to the final ligation step. This is usually performed before setting up the annealing reactions. In brief, the “universal” oligonucleotides encoding the constant regions of the proteins (oligos 1, 2 and 3 of above) are annealed with their complementary oligonucleotides. Additionally, the “specific” oligonucleotides encoding the finger recognition helices are annealed with their respective complementary oligonucleotides. These complementary oligos are designed to fill in the region that was previously filled in by polymerase in the above-mentioned protocol. Oligonucleotides complementary to oligos 1 and 6 are engineered to leave overhanging sequences specific for the restriction sites used in cloning into the vector of choice in the following step. The second assembly protocol differs from the initial protocol in the following aspects: the “scaffold” encoding the newly designed ZFP is composed entirely of synthetic DNA thereby eliminating the polymerase fill-in step, additionally the fragment to be cloned into the vector does not require amplification. Lastly, the design of leaving sequence-specific overhangs eliminates the need for restriction enzyme digests of the inserting fragment. Alternatively, changes to ZFP recognition helices can be created using conventional site-directed mutagenesis methods.

Both assembly methods require that the resulting fragment encoding the newly designed ZFP be ligated into a vector. Ultimately, the ZFP-encoding sequence is cloned into an expression vector. Expression vectors that are commonly utilized include, but are not limited to, a modified pMAL-c2 bacterial expression vector (New England BioLabs, Beverly, Mass.) or an eukaryotic expression vector, pcDNA (Promega, Madison, Wis.). The final constructs are verified by sequence analysis.

Any suitable method of protein purification known to those of skill in the art can be used to purify ZFPs (see, Ausubel, supra, Sambrook, supra). In addition, any suitable host can be used for expression, e.g., bacterial cells, insect cells, yeast cells, mammalian cells, and the like.

Expression of a zinc finger protein fused to a maltose binding protein (MBP-ZFP) in bacterial strain JM109 allows for straightforward purification through an amylose column (New England BioLabs, Beverly, Mass.). High expression levels of the zinc finger chimeric protein can be obtained by induction with IPTG since the MBP-ZFP fusion in the pMal-c2 expression plasmid is under the control of the tac promoter (New England BioLabs, Beverly, Mass.). Bacteria containing the MBP-ZFP fusion plasmids are inoculated into 2xYT medium containing 10 μM ZnCl₂, 0.02% glucose, plus 50 μg/ml ampicillin and shaken at 37° C. At mid-exponential growth IPTG is added to 0.3 mM and the cultures are allowed to shake. After 3 hours the bacteria are harvested by centrifugation, disrupted by sonication or by passage through a pressure cell or through the use of lysozyme, and insoluble material is removed by centrifugation. The MBP-ZFP proteins are captured on an amylose-bound resin, washed extensively with buffer containing 20 mM Tris-HCl (pH 7.5), 200 mM NaCl, 5 mM DTT and 50.mu.M ZnCl₂, then eluted with maltose in essentially the same buffer (purification is based on a standard protocol from New England BioLabs. Purified proteins are quantitated and stored for biochemical analysis.

The dissociation constant of a purified protein, e.g., Kd, is typically characterized via electrophoretic mobility shift assays (EMSA) (Buratowski & Chodosh, in Current Protocols in Molecular Biology pp. 12.2.1-12.2.7 (Ausubel ed., 1996)). Affinity is measured by titrating purified protein against a fixed amount of labeled double-stranded oligonucleotide target. The target typically comprises the natural binding site sequence flanked by the 3 bp found in the natural sequence and additional, constant flanking sequences. The natural binding site is typically 9 bp for a three-finger protein and 2.times.9 bp+intervening bases for a six finger ZFP. The annealed oligonucleotide targets possess a 1 base 5′ overhang that allows for efficient labeling of the target with T4 phage polynucleotide kinase. For the assay the target is added at a concentration of 1 nM or lower (the actual concentration is kept at least 10-fold lower than the expected dissociation constant), purified ZFPs are added at various concentrations, and the reaction is allowed to equilibrate for at least 45 min. In addition the reaction mixture also contains 10 mM Tris (pH 7.5), 100 mM KCl, 1 mM MgCl₂, 0.1 mM ZnCl₂, 5 mM DTT, 10% glycerol, 0.02% BSA.

The equilibrated reactions are loaded onto a 10% polyacrylamide gel, which has been pre-run for 45 min in Tris/glycine buffer, then bound and unbound labeled target is resolved by electrophoresis at 150V. Alternatively, 10-20% gradient Tris-HCl gels, containing a 4% polyacrylamide stacking gel, can be used. The dried gels are visualized by autoradiography or phosphorimaging and the apparent Kd is determined by calculating the protein concentration that yields half-maximal binding.

The assays can also include a determination of the active fraction in the protein preparations. Active fraction is determined by stoichiometric gel shifts in which protein is titrated against a high concentration of target DNA. Titrations are done at 100, 50, and 25% of target (usually at micromolar levels).

The technique of phage display provides a largely empirical means of generating zinc finger proteins with desired target specificity (see e.g., Rebar, U.S. Pat. No. 5,789,538; Choo et al., WO 96/06166; Barbas et al., WO 95/19431 and WO 98/543111.

### B. Regulatory Domains

Zinc finger proteins are often expressed with an exogenous domain (or functional fragment thereof) as fusion proteins. Common domains for addition to the ZFP include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases, topoisomerases, helicases, ligases, kinases, phosphatases, polymerases, endonucleases) and their associated factors and modifiers. A preferred domain for fusing with a ZFP when the ZFP is to be used for repressing expression of a target gene is a KRAB repression domain from the human KOX-1 protein (Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994). Preferred domains for achieving activation include the HSV VP16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Seifpal et al., EMBO J. 11, 4961-4968 (1992)).

The identification of novel sequences and accessible regions (e.g., DNase I hypersensitive sites) in genes involved in neuropathic pain allows for the design of fusion molecules for the treatment of pain. Thus, in certain embodiments, the compositions and methods disclosed herein involve fusions between a DNA-binding domain specifically targeted to one or more regulatory regions of a target gene involved in neuropathic pain and a functional (e.g., repression or activation) domain (or a polynucleotide encoding such a fusion). In this way, the repression or activation domain is brought into proximity with a sequence in the gene that is bound by the DNA-binding domain. The transcriptional regulatory function of the functional domain is then able to act on the selected regulatory sequences.

In additional embodiments, targeted remodeling of chromatin, as disclosed in co-owned WO 01/83793 can be used to generate one or more sites in cellular chromatin that are accessible to the binding of a DNA binding molecule.

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, Ill.) Catalogue. Methods and compositions for making fusions between a minor groove binder and a polypeptide have been described. Mapp et al. (2000) Proc. Natl. Acad. Sci. USA 97:3930-3935.

In certain embodiments, the target site bound by the zinc finger protein is present in an accessible region of cellular chromatin. Accessible regions can be determined as described, for example, in co-owned International Publication WO 01/83732. If the target site is not present in an accessible region of cellular chromatin, one or more accessible regions can be generated as described in co-owned WO 01/83793. In additional embodiments, the DNA-binding domain of a fusion molecule is capable of binding to cellular chromatin regardless of whether its target site is in an accessible region or not. For example, such DNA-binding domains are capable of binding to linker DNA and/or nucleosomal DNA. Examples of this type of “pioneer” DNA binding domain are found in certain steroid receptor and in hepatocyte nuclear factor 3 (HNF3). Cordingley et al. (1987) Cell 48:261-270; Pina et al. (1990) Cell 60:719-731; and Cirillo et al. (1998) EMBO J. 17:244-254.

For such applications, the fusion molecule is typically formulated with a pharmaceutically acceptable carrier, as is known to those of skill in the art. See, for example, Remington's Pharmaceutical Sciences, 17th ed., 1985; and co-owned WO 00/42219.

The functional component/domain of a fusion molecule can be selected from any of a variety of different components capable of influencing transcription of a gene once the fusion molecule binds to a target sequence via its DNA binding domain. Hence, the functional component can include, but is not limited to, various transcription factor domains, such as activators, repressors, co-activators, co-repressors, and silencers.

Exemplary functional domains for fusing with a DNA-binding domain such as, for example, a ZFP, to be used for repressing expression of a gene is a KOX repression domain or a KRAB repression domain from the human KOX-1 protein (see, e.g., Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994). Another suitable repression domain is the repression domain from TGF-beta inducible early gene (TIEG) (Cook et al (1999) J.B.C 274(41):29500-29504). Another useful repression domain is that associated with the v-ErbA protein. See, for example, Damm, et al. (1989) Nature 339:593-597; Evans (1989) Int. J. Cancer Suppl. 4:26-28; Pain et al. (1990) New Biol. 2:284-294; Sap et al. (1989) Nature 340:242-244; Zenke et al. (1988) Cell 52:107-119; and Zenke et al. (1990) Cell 61:1035-1049.

Suitable domains for achieving activation include the HSV VP16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Seifpal et al., EMBO J. 11, 4961-4968 (1992)), and Degron (Molinari et al (1999) EMBO J. 18(22):6439-6447). Additional exemplary activation domains include, but are not limited to, VP16, VP64, p300, CBP, PCAF, SRC1 PvALF, AtHD2A and ERF-2. See, for example, Robyr et al. (2000) Mol. Endocrinol. 14:329-347; Collingwood et al. (1999) J. Mol. Endocrinol. 23:255-275; Leo et al. (2000) Gene 245: 1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, AP1, ARF-5, -6, -7, and -8, CPRF1, CPRF4, MYC-RP/GP, and TRAB1. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15,353.

Additional exemplary repression domains include, but are not limited to, KRAB (also referred to as “KOX”), SID, MBD2, MBD3, members of the DNMT family (e.g., DNMT1, DNMT3A, DNMT3B), Rb, and MeCP2. See, for example, Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

Additional exemplary functional domains are disclosed, for example, in co-owned U.S. Pat. No. 6,534,261 and US Patent Application Publication No. 2002/0160940.

### C. Expression Vectors

The nucleic acid encoding the ZFP of choice is typically cloned into intermediate vectors for transformation into prokaryotic or eukaryotic cells for replication and/or expression, e.g., for determination of Kd. Intermediate vectors are typically prokaryote vectors, e.g., plasmids, or shuttle vectors, or insect vectors, for storage or manipulation of the nucleic acid encoding ZFP or production of protein. The nucleic acid encoding a ZFP is also typically cloned into an expression vector, for administration to a plant cell, animal cell, preferably a mammalian cell or a human cell, fungal cell, bacterial cell, or protozoal cell.

To obtain expression of a cloned gene or nucleic acid, a ZFP is typically subcloned into an expression vector that contains a promoter to direct transcription. Suitable bacterial and eukaryotic promoters are well known in the art and described, e.g., in Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). Bacterial expression systems for expressing the ZFP are available in, e.g., E. coli, Bacillus sp., and Salmonella (Palva et al., Gene 22:229-235 (1983)). Kits for such expression systems are commercially available. Eukaryotic expression systems for mammalian cells, yeast, and insect cells are well known in the art and are also commercially available.

The promoter used to direct expression of a ZFP nucleic acid depends on the particular application. For example, a strong constitutive promoter is typically used for expression and purification of ZFP. In contrast, when a ZFP is administered in vivo for gene regulation, either a constitutive or an inducible promoter is used, depending on the particular use of the ZFP. In addition, a preferred promoter for administration of a ZFP can be a weak promoter, such as HSV TK or a promoter having similar activity. The promoter typically can also include elements that are responsive to transactivation, e.g., hypoxia response elements, Gal4 response elements, lac repressor response elements, and small molecule control systems such as tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the ZFP, and signals required, e.g., for efficient polyadenylation of the transcript, transcriptional termination, ribosome binding sites, or translation termination. Additional elements of the cassette may include, e.g., enhancers, and exogenous spliced intronic signals.

The particular expression vector used to transport the genetic information into the cell is selected with regard to the intended use of the ZFP. Standard bacterial expression vectors include plasmids such as pBR322 based plasmids, pSKF, pET23D, and commercially available fusion expression systems such as GST and LacZ. A preferred fusion protein is the maltose binding protein, “MBP.” Such fusion proteins are used for purification of the ZFP. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with a ZFP encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, naked DNA, plasmid vectors, viral vectors, both episomal and integrative, and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

### VII. ASSAYS

Once a ZFP has been designed and prepared according to the procedures just set forth, an initial assessment of the activity of the designed ZFP is undertaken. ZFP proteins showing the ability to modulate the expression of a gene of interest can then be further assayed for more specific activities depending upon the particular application for which the ZFPs have been designed. Thus, for example, the ZFPs provided herein can be initially assayed for their ability to modulate expression of genes involved in neuropathic pain. More specific assays of the ability of the ZFP to modulate expression of the target genes involved in neuropathic pain to treat this pain are then typically undertaken. A description of these more specific assays are set forth infra in section IX.

The activity of a particular ZFP can be assessed using a variety of in vitro and in vivo assays, by measuring, e.g., protein or mRNA levels, product levels, enzyme activity, tumor growth; transcriptional activation or repression of a reporter gene; second messenger levels (e.g., cGMP, cAMP, IP3, DAG, Ca2+); cytokine and hormone production levels; and neovascularization, using, e.g., immunoassays (e.g., ELISA and immunohistochemical assays with antibodies), hybridization assays (e.g., RNase protection, Northerns, in situ hybridization, oligonucleotide array studies), colorimetric assays, amplification assays, enzyme activity assays, tumor growth assays, phenotypic assays, and the like.

ZFPs are typically first tested for activity in vitro using cultured cells, e.g., 293 cells, CHO cells, VERO cells, BHK cells, HeLa cells, COS cells, and the like. Preferably, human cells are used. The ZFP is often first tested using a transient expression system with a reporter gene, and then regulation of the target endogenous gene is tested in cells and in animals, both in vivo and ex vivo. The ZFP can be recombinantly expressed in a cell, recombinantly expressed in cells transplanted into an animal, or recombinantly expressed in a transgenic animal, as well as administered as a protein to an animal or cell using delivery vehicles described below. The cells can be immobilized, be in solution, be injected into an animal, or be naturally occurring in a transgenic or non-transgenic animal.

Modulation of gene expression is tested using one of the in vitro or in vivo assays described herein. Samples or assays are treated with a ZFP and compared to untreated control samples to examine the extent of modulation. As described above, for regulation of endogenous gene expression, the ZFP typically has a Kd of 200 nM or less, more preferably 100 nM or less, more preferably 50 nM, and most preferably 25 nM or less.

The effects of the ZFPs can be measured by examining any of the parameters described above. Any suitable gene expression, phenotypic, or physiological change can be used to assess the influence of a ZFP. When the functional consequences are determined using intact cells or animals, one can also measure a variety of effects such as neurotrophism, transcriptional changes to both known and uncharacterized genetic markers (e.g., Northern blots or oligonucleotide array studies), changes in cell metabolism such as cell growth or pH changes, and changes in intracellular second messengers such as cAMP or cGMP.

Preferred assays for ZFP regulation of endogenous gene expression can be performed in vitro. In one preferred in vitro assay format, ZFP regulation of endogenous gene expression in cultured cells is measured by examining protein production using an ELISA assay. The test sample is compared to control cells treated with a vector lacking ZFP-encoding sequences or a vector encoding an unrelated ZFP that is targeted to another gene.

In another embodiment, ZFP regulation of endogenous gene expression is determined in vitro by measuring the level of gene mRNA expression (e.g., expression level of Nav1.8 gene). The level of gene expression is measured using amplification, e.g., using PCR, LCR, or hybridization assays, e.g., Northern hybridization, dot blotting and RNase protection. The use of quantitative RT-PCR techniques (i.e., the so-called TaqMan® assays) can also be utilized to quantitate the level of transcript. The level of protein or mRNA is detected using directly or indirectly labeled detection agents, e.g., fluorescently or radioactively labeled nucleic acids, radioactively or enzymatically labeled antibodies, and the like, as described herein. Such methods are also described in U.S. Pat. Nos. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995), each of which is incorporated by reference in its entirety.

Alternatively, a reporter gene system can be devised using a gene promoter from the selected target gene (e.g., Nav1.8) operably linked to a reporter gene such as luciferase, green fluorescent protein, CAT, GAPDH, β-gal, etc. The reporter construct is typically co-transfected into a cultured cell. After treatment with the ZFP of choice, the amount of reporter gene transcription, translation, or activity is measured according to standard techniques known to those of skill in the art.

Another example of a preferred assay format useful for monitoring ZFP regulation of endogenous gene expression is performed in vivo. This assay is particularly useful for examining genes involved in chronic pain. In this assay, the ZFP of choice is administered (e.g., via intramuscular or intravenous injection) into an animal exhibiting aberrant nerve excitability. After a suitable length of time, preferably 4-8 weeks, nerve function and/or gene expression are compared to control animals that also have aberrant nerve excitability but did not receive a ZFP. Nerve excitability that is significantly different as between control and test animals (using, e.g., Student's T test) are determined to have been affected by the ZFP.

### VIII. PHARMACEUTICAL COMPOSITIONS

The ZFPs provided herein, and more typically the nucleic acids encoding them, can optionally be formulated with a pharmaceutically acceptable carrier as a pharmaceutical composition.

### A. Nucleic Acid Based Compositions

Conventional viral and non-viral based gene transfer methods can be used to introduce nucleic acids encoding the present ZFPs into mammalian cells or target tissues. Such methods can be used to administer nucleic acids encoding ZFPs to cells in vitro. In some instances, the nucleic acids encoding ZFPs are administered for in vivo or ex vivo gene therapy uses. Non-viral vector delivery systems include DNA plasmids, naked nucleic acid, and nucleic acid complexed with a delivery vehicle such as poloxamers or liposomes. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11: 167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

Methods of non-viral delivery of nucleic acids encoding the ZFPs provided herein include lipofection, microinjection, biolistics, virosomes, liposomes, immunoliposomes, polycation or lipid:nucleic acid conjugates, naked DNA, artificial virions, electroporation and agent-enhanced uptake of DNA. Lipofection is described in, e.g., U.S. Pat. Nos. 5,049,386, 4,946,787; and 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids encoding engineered ZFPs take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of ZFPs can include retroviral, lentivirus, adenoviral, adeno-associated and herpes simplex virus (HSV) vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long-term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system can therefore depend on the target tissue. Retroviral vectors are comprised of cis-acting long terminal repeats (LTRs) with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression of the ZFP is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) represent another alternative gene delivery system based on the defective and nonpathogenic parvovirus adeno-associated virus. All vectors are derived from a plasmid that retains only the AAV 145 bp inverted terminal repeats flanking the transgene expression cassette. Efficient gene transfer and long-term transgene expression are key features for this vector system. (Wagner et al., Lancet 351:9117 1702-3 (1998), Kearns et al., Gene Ther. 9:748-55 (1996)). Other AVV serotypes, including AAV1 to AAV8, can also be used in accordance with the present invention.

Replication-deficient recombinant adenoviral vectors (Ad) are predominantly used for colon cancer gene therapy because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:15-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topfet al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and .psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome that are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

As stated above, various viral delivery vehicles, as are known in the art, can be used to introduce a nucleic acid (e.g., a nucleic acid encoding a zinc finger protein) into a cell. The choice of delivery vehicle depends upon a number of factors, including but not limited to the size of the nucleic acid to be delivered and the desired target cell.

In certain embodiments, adenoviruses are used as delivery vehicles. Exemplary adenovirus vehicles include Adenovirus Types 2, 5, 12 and 35. For example, vehicles useful for introduction of transgenes into hematopoietic stem cells, e.g., CD34+ cells, include adenovirus Type 35. Additional adenoviral vehicles include the so-called “gutless” adenoviruses. See, for example, Kochanek et al. (1996) Proc. Natl. Acad. Sci. USA 93:5,731-5,736.

Lentivirus delivery vehicles have been described, for example, in U.S. Pat. Nos. 6,312,682 and 6,669,936 and in U.S. Patent Application Publication No. 2002/0173030 and can be used, e.g., to introduce transgenes into immune cells (e.g., T-cells). Lentiviruses are capable of integrating a DNA copy of their RNA genome into the genome of a host cell. See, for example, Ory et al. (1996) Proc. Natl. Acad. Sci. USA 93:11382-11388; Miyoshi et al. (1998) J. Virology 72:8150-8157; Dull et al. (1998) J. Virol. 72:8463-8471; Zuffery et al. (1998) J. Virol. 72:9873-9880; Follenzi et al. (2000) Nature Genetics 25:217-222 and Delenda (2004) J. Gene Medicine 6:S125-S138. In certain lentiviral vehicles, this integration function has been disabled to generate non-integrating lentivirus vehicles. See, for example, Poon et al. (2003) J. Virology 77:3962-3972 and Vargas et al. (2004) Human Gene Therapy 15:361-372. The use of both integrating and non-integrating lentivirus vectors for transduction of hematopoietic stem cells has been described by Haas et al. (2000) Mol. Therapy. 2:71-80. Transduction of CD4+ T-cells with integrating lentivirus vectors has been described by Humeau et al. (2004) Mol. Therapy. 9:902-913.

Herpes simplex virus vehicles, which are capable of long-term expression in neurons and ganglia, have been described. See, for example, Krisky et al. (1998) Gene Therapy 5(11):1517-1530; Krisky et al. (1998) Gene Therapy 5(12):1593-1603; Burton et al. (2001) Stem Cells 19:358-377; Lilley et al. (2001) J. Virology 75(9):4343-4356

Methods for improving the efficiency of retroviral transduction of hematopoietic stem cells are disclosed, for example, in U.S. Pat. No. 5,928,638.

The tropism of retroviral and lentiviral delivery vehicles can be altered by the process of pseudotyping, thereby enabling viral delivery of a nucleic acid to a particular cell type. See, for example, U.S. Pat. No. 5,817,491.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses' outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In some instances, cells are isolated from the subject organism, transfected with a ZFP nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+(panB cells), GR-1 (granulocytes), and lad (differentiated antigen presenting cells) (see Inaba et al., J. Exp. Med. 176:1693-1702 (1992)).

Vectors (e.g., retroviruses, adenoviruses, liposomes, etc.) containing therapeutic ZFP nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions, as described below (see, e.g., Remington's Pharmaceutical Sciences, 17th ed., 1989).

### B. Protein Compositions

An important factor in the administration of polypeptide compounds, such as the present ZFPs, is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides such as ZFPs across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996)). Another subsequence, the h (hydrophobic) domain of signal peptides, was found to have similar cell membrane translocation characteristics (see, e.g., Lin et al., J. Biol. Chem. 270:14255-14258 (1995)).

Examples of peptide sequences which can be linked to a ZFP, for facilitating uptake of ZFP into cells, include, but are not limited to: an 11 amino acid peptide of the tat protein of HIV; a 20 residue peptide sequence which corresponds to amino acids 84-103 of the p16 protein (see Fahraeus et al., Current Biology 6:84 (1996)); the third helix of the 60-amino acid long homeodomain of Antennapedia (Derossi et al., J. Biol. Chem. 269:10444 (1994)); the h region of a signal peptide such as the Kaposi fibroblast growth factor (K-FGF) h region (Lin et al., supra); or the VP22 translocation domain from HSV (Elliot & O'Hare, Cell 88:223-233 (1997)). Other suitable chemical moieties that provide enhanced cellular uptake may also be chemically linked to ZFPs. Membrane translocation domains (i.e., internalization domains) can also be selected from libraries of randomized peptide sequences. See, for example, Yeh et al. (2003) Molecular Therapy 7(5):S461, Abstract #1191.

Toxin molecules also have the ability to transport polypeptides across cell membranes. Often, such molecules are composed of at least two parts (called “binary toxins”): a translocation or binding domain or polypeptide and a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used in attempts to deliver peptides to the cell cytosol as internal or amino-terminal fusions (Arora et al., J. Biol. Chem., 268:3334-3341 (1993); Perelle et al., Infect. Immun., 61:5147-5156 (1993); Stemnark et al., J. Cell Biol. 113:1025-1032 (1991); Donnelly et al., PNAS 90:3530-3534 (1993); Carbonetti et al., Abstr. Annu. Meet. Am. Soc. Microbiol. 95:295 (1995); Sebo et al., Infect. Immun. 63:3851-3857 (1995); Klimpel et al., PNAS U.S.A. 89:10277-10281 (1992); and Novak et al., J. Biol. Chem. 267:17186-17193 1992)).

Such subsequences can be used to translocate ZFPs across a cell membrane. ZFPs can be conveniently fused to or derivatized with such sequences. Typically, the translocation sequence is provided as part of a fusion protein. Optionally, a linker can be used to link the ZFP and the translocation sequence. Any suitable linker can be used, e.g., a peptide linker.

The ZFP can also be introduced into an animal cell, preferably a mammalian cell, via liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell, i.e., a ZFP. The liposome fuses with the plasma membrane, thereby releasing the drug into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (in this case, a ZFP) at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane (see, e.g., PNAS 84:7851 (1987); Biochemistry 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

Such liposomes typically comprise a ZFP and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91.backslash.17424, Deamer & Bangham, Biochim. Biophys. Acta 443:629-634 (1976); Fraley, et al., PNAS 76:3348-3352 (1979); Hope et al., Biochim. Biophys. Acta 812:55-65 (1985); Mayer et al., Biochim. Biophys. Acta 858:161-168 (1986); Williams et al., PNAS 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

In some instances, liposomes are targeted using targeting moieties that are specific to a particular cell type, tissue, and the like. Targeting of liposomes using a variety of targeting moieties (e.g., ligands, receptors, and monoclonal antibodies) has been previously described (see, e.g., U.S. Pat. Nos. 4,957,773 and 4,603,044).

Standard methods for coupling targeting agents to liposomes can be used. These methods generally involve incorporation into liposome lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A (see Renneisen et al., J. Biol. Chem., 265:16337-16342 (1990) and Leonetti et al., PNAS 87:2448-2451 (1990).

### C. Dosage

For therapeutic applications of ZFPs, the dose administered to a patient should be sufficient to affect a beneficial therapeutic response in the patient over time. The dose will be determined by the efficacy and Kd of the particular ZFP employed, the nuclear volume of the target cell, and the condition of the patient, as well as the body weight or surface area of the patient to be treated. The size of the dose also will be determined by the existence, nature, and extent of any adverse side effects that accompany the administration of a particular compound or vector in a particular patient.

In determining the effective amount of the ZFP to be administered in the treatment or prophylaxis of neuropathic pain, the physician evaluates circulating plasma levels of the ZFP or nucleic acid encoding the ZFP, potential ZFP toxicities, progression of the disease, and the production of anti-ZFP antibodies. Administration can be accomplished via single or divided doses.

### D. Compositions and Modes of Administration

### 1. General

ZFPs and the nucleic acids encoding the ZFPs can be administered directly to a subject (e.g., patient) for modulation of gene expression and for therapeutic or prophylactic applications. In general, and in view of the discussion herein, phrases referring to introducing a ZFP into an animal or patient can mean that a ZFP or ZFP fusion protein is introduced and/or that a nucleic acid encoding a ZFP or ZFP fusion protein is introduced in a form that can be expressed in the animal. For example, as described in greater detail in the following section, the ZFPs and/or nucleic acids can be used in the treatment of chronic pain.

Administration of therapeutically effective amounts is by any of the routes normally used for introducing ZFP into ultimate contact with the tissue to be treated. The ZFPs are administered in any suitable manner, preferably with pharmaceutically acceptable carriers (e.g., poloxamer and/or buffer). Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there are a wide variety of suitable formulations of pharmaceutical compositions (see, e.g., Remington's Pharmaceutical Sciences, 17th ed. 1985)).

The ZFPs, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of the disclosed methods, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

### 2. Exemplary Delivery Options

A variety of delivery options are available for the delivery of the pharmaceutical compositions provided herein so as to modulate expression of genes involved in neuropathic pain. Depending upon the particular indication (e.g., which nerve(s) involved in the pain), the compositions can be targeted to specific areas or tissues of a subject. For example, in some methods, one delivers compositions to specific regions of the body to treat pain. Other treatments, in contrast, involve administering the composition in a general manner without seeking to target delivery to specific regions.

A number of approaches can be utilized to localize the delivery of agents to particular regions. Certain of these methods involve delivery to the body lumen or to a tissue (see, e.g., U.S. Pat. Nos. 5,941,868; 6,067,988; 6,050,986; and 5,997,509; as well as PCT Publications WO 00/25850; WO 00/04928; 99/59666; and 99/38559). Options for the delivery of compositions to modulate genes involved in neuropathic pain include systemic administration using intravenous or subcutaneous administration, and tissue engineering (U.S. Pat. No. 5,944,754). Various vectors can be used to deliver polynucleotides to sensory neurons and/or ganglia. See, e.g., Glorioso et al. (2003) Curr Opin Mol. Ther. 5(5):483-488. See also Fleming et al. (2001) Hum Gene Ther. 12(1):77-86; Goins et al. (1999) J. Virol. 73(1):519-532; Xu et al. (2003) Proc Natl Acad Sci USA 100(10):6204-6209 and Glatzel et al. (2000) Proc Natl Acad Sci USA 97(1):442-447.

Other delivery methods known by those skilled in the art include the methods disclosed in U.S. Pat. Nos. 5,698,531; 5,893,839; 5,797,870; 5,693,622; 5,674,722; 5,328,470; and 5,707,969.

### IX. APPLICATIONS

### A. General

ZFPs engineered to bind a chosen target site in a gene of interest, and nucleic acids encoding them, can be utilized to modulate expression of a target gene (e.g., genes involved in neuropathic pain) in any subject and by so doing, treat neuropathic pain. Generally, a target site of a nucleic acid within a cell or population of cells is contacted with a ZFP that has binding specificity for that target site. Methods can be performed in vitro with cell cultures or in vivo. Certain methods are performed such that chronic pain is treated by repressing expression of one or more genes involved hyper-excitability (e.g., Nav1.8).

### B. Transgenic/knockout Animals

Using the compositions and methods described herein, transgenic animals can be generated using standard techniques. For example, the vectors containing the DNA segments of interest can be transferred into a host cell via calcium phosphate treatment, electroporation, lipofection, biolistics or viral-based transduction. Other methods used to transform mammalian cells include the use of polybrene, protoplast fusion, liposomes, electroporation, and microinjection (see generally, Sambrook et al., Molecular Cloning: A Laboratory Manual (C.S.H.P. Press, NY 2d ed., 1989)). For production of transgenic animals, transgenes can be microinjected into fertilized oocytes, or can be incorporated into the genome of embryonic stem cells and the nuclei of such cells transferred into enucleated oocytes. In addition, gene knockouts (e.g., Nav1.8) or knockdowns can also be generated. For example, a ZFP as described herein, which is targeted to one or more genes involved in neuropathic pain, is administered to any animal in order to create a knockout or knockdown animal.

These animals are useful as models for disease and for drug testing. Thus, ZFP repressors as described herein make it possible to reduce or eliminate gene (e.g., Nav1.8) activity in any animal model, for which no feasible methods currently exist to generate knockouts. Furthermore, as many accepted animal models for studying chronic pain and evaluating candidate drugs are non-mouse models, the ability to create these knockouts/knockdowns in any animal using the ZFPs described herein represents an important advance in the field. Because ZFP-mediated Nav1.8 repression interferes with pain signaling in vivo, transgenic animals treated with the ZFP repressors of Nav1.8, as described herein, will be less sensitive to a variety of pain stimuli. Such methods can be used to improve the well-being of animals used in biomedical research.

In addition, animal models for drug screening can be generated by using ZFPs comprising a transcriptional activation domain to up-regulate expression of, e.g., a Nav1.8 gene.

### C. Therapeutic Applications

The ZFPs provided herein and the nucleic acids encoding them, such as in the pharmaceutical compositions described herein, can be utilized to modulate (e.g., activate or repress) expression of one or more genes involved in nerve excitability, thereby modulating chronic pain. Modulation of nerve excitability can result in the amelioration or elimination of chronic pain. For example, genes overexpressed in chronic pain can be repressed using targeted ZFPs both in cell cultures (i.e., in in vitro applications) and in vivo to decrease nerve hyper-excitability and thereby treat chronic pain. Unlike the antisense approach, which needs to target a large number of copies of mRNA, there are a limited number of binding sites in each cell to be targeted by a ZFP, i.e., the chromosomal copies of the target gene(s), therefore, ZFPs can function at a relatively low expression level.

Hence, certain methods for treating chronic pain involve introducing a ZFP targeted to Nav1.8 into an animal. Binding of the ZFP bearing a repression domain to its target site results in decreased nerve excitability and amelioration (or elimination) of neuropathic pain. Typically, a repression domain fused to the ZFP represses the expression of the target gene.

A variety of assays for assessing gene expression as it relates to nerve excitability and pain are known. For example, electrophysiological recordings (e.g., to determine hyper-excitability and/or spontaneous activity) can be obtained. See, e.g., Liu et al. (2001) Neuroscience 105(1):265-75; Cain et al. (2001) J. Neurosci. 21(23):9367-76. Heat sensitization can also be measured. Other options that may be used alone or in combination with any of the above assay methods are immunostaining of nerves and/or of overlying tissue (e.g., skin), for example to determine morphological changes (e.g., branching, decrease in fibers, etc). In addition, microscopic examination of tissue sections can be performed. These and other methods are accepted assays and the results can also be extrapolated to other systems.

Additional assays are described, for example, by Lutfy et al. (1997) Pain 70(1):31-40; Foo et al. (1993) Pharmacol Biochem Behav 45(2):501-505 and Eaton et al. (2002) Gene Ther. 9(20):1387-1395.

### D. Cell Culture Models for Drug Screening and Validation

Because no human transformed cell line expresses a significant level of Nav1.8, screening for inhibitors of Nav1.8 is difficult in cell culture without using cDNA-mediated overexpression. However, cDNA leads to a high and non-physiological level of expression that is not ideal for identifying drug molecules that will be reactive against the physiological level of Nav1.8. ZFP activators in accordance with the present invention can be used to induce the expression of the endogenous Nav1.8 gene in cultured cells and provide a more physiologically relevant level of Nav1.8 for high-through-put screening to identify and/or to validate Nav1.8 inhibitors.

The following examples are provided solely to illustrate in greater detail particular aspects of the disclosed methods and compositions and should not be construed to be limiting in any way.

### Example 1

### Materials and Methods

### A. Zinc Finger Protein Transcription Factor 8982-KRAB

ZFP-TF 8982-KRAB consists of a nuclear localization sequence (PKKKRKV (SEQ ID NO:43)) from SV40 large T antigen, an engineered zinc finger DNA-binding domain targeted to the Nav1.8 gene, a KRAB A/B repression domain from KOX1 transcription factor (amino acids 1 to 98), and a flag-epitope tag (DYKDDDDK (SEQ ID NO:44)). The designed DNA-binding domain contains six finger modules, each comprising the composition of amino acids as set forth in Table 1 in reference to ZFP “8982”.

### B. Cell Culture, Transfection and Transduction

Human DAOY cells (ATCC) were cultured in DMEM supplemented with 10% FBS. The DAOY cells were transduced with lentiviral vectors encoding REST-p65, GFP or a zinc finger protein transcription factor (ZFP-TF 8982-KRAB), as described below.

A self-inactivating HIV-directed vector RRL (see Dull et al. (1998) J Virol 72(11):8463-8471), containing the woodchuck hepatitis post-transcriptional regulatory element (WPRE) and a polyurine tract, was modified to carry the appropriate transgene expression cassette under the control of the CMV promoter. Lentiviral vectors were prepared by transient transfection of 293T cells with 4 plasmids (see Tiscomia et al. (2006) Nat Protoc 1(1):241-245), the lentiviral transfer vector for specific transgene expression (i.e., REST-p65, GFP, or ZFP-TF 8982-KRAB), and 3 additional packaging constructs pMDL, pREV and pVSV-G (Invitrogen) using Lipofectamine 2000 (Invitrogen) per manufacturer's instructions. Transfection medium was changed to growth medium 16 hours following transfection. The virus containing media were then collected after culturing for an additional 24 and 48 hours and centrifuged at 3000 rpm for 10 min. The supernatant was filtered through a 0.22 μm filter and concentrated 150-fold by ultracentrifugation at 28,000 rpm for 2 hours. Viral stocks were then made in small aliquots and stored at −70° C.

Lentiviral vectors were titered by limited serial dilution of viral stocks on 293T cells, followed by analyzing the number of proviral DNA copies per cell by real-time quantitative PCR using Taqman® chemistry in 96-well format on an ABI 7700 SDS machine (Perkin Elmer) as described by Liu et al. (2001) J Biol Chem 276(14):11323-11334. The relative amount of proviral DNA from transfected cells was normalized to a house-keeping gene albumin. This number was then converted to the number of proviral DNA copies using lentiviral vector encoding GFP as a standard. Proviral and albumin DNA were quantified using proviral DNA primer/probe set (CCAACGAAGACAAGATCTGC (SEQ ID NO: 45), TCCTGCGTCGAGAGAGCT (SEQ ID NO: 46), and FAM-CGCCCGAACAGGGACCTGAAAGC-BHQ1 (SEQ ID NO: 47)) and albumin primer/probe set (TGAAACATACGTTCCCAAAGAGTTT (SEQ ID NO: 48), CTCTCCTTCTCAGAAAGTGTGCATAT (SEQ ID NO: 49), and FAM-TGCTGAAACATTCACCTTCCATGCAGA-BHQ1 (SEQ ID NO: 50)) respectively. The transduction titer of lentiviral vector encoding GFP was determined by flow cytometric analysis after limited serial dilution on 293T cells.

REST-p65 expression constructs were assembled as a fusion consisting of the nuclear localization sequence (PKKKRKV (SEQ ID NO: 43)) from SV40 large T antigen, the DNA-binding domain from human RE1-silencing transcription factor (amino acids 152-440) (see Ooi et al. (2006) J Biol Chem 281(51):38974-38980), the p65 transcriptional activation domain from human NF-κB (amino acids 288-548) (see Ruben et al. (1991) Science 251(5000):1490-1493), and a flag epitope tag (DYKDDDDK (SEQ ID NO: 44)). The entire REST-p65 expression cassette was cloned into the lentiviral transfer vector RRL and was used to prepare lentiviral vector driving expression of REST-p65.

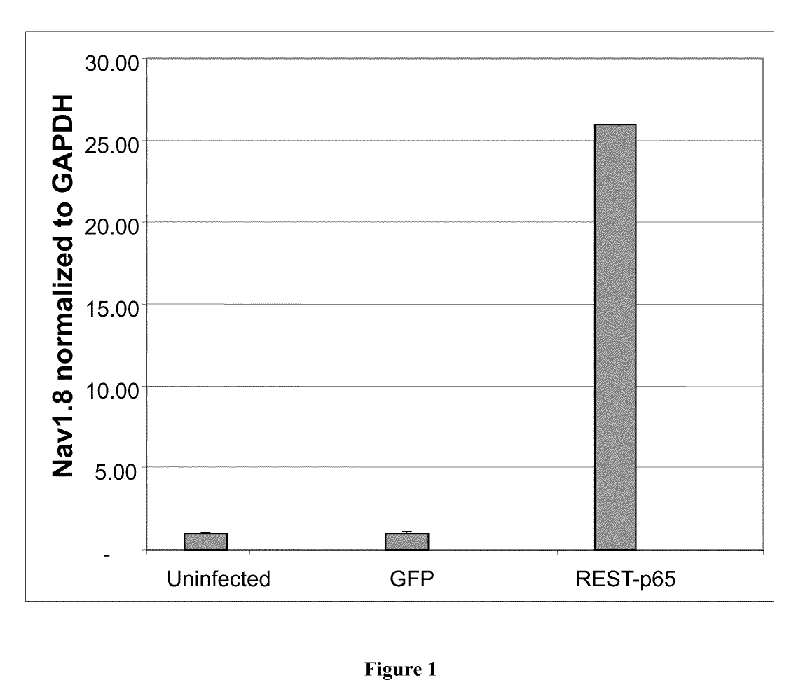

Human DAOY cells were transduced with lentiviral vectors encoding REST-p65 or GFP at 10 MOI for 3 days. Because Nav1.8 is expressed almost exclusively in the peripheral sensory nervous system (dorsal root ganglia and the sciatic nerve) wherein high levels of Nav1.8 are expressed, a cell culture model having such increased Nav1.8 expression was desirable for testing ZFP repressors of Nav1.8. FIG. 1 illustrates the increased expression (˜25 fold) of Nav1.8 in such cells, normalized to human GAPDH mRNA, as compared to untransfected and GFP controls.

Human DAOY cells were transduced with lentiviral vectors encoding REST-p65 at 10 MOI to elevate the basal level of Nav1.8 gene expression (as described above in reference to FIG. 1). The elevated level of Nav1.8 mRNA stabilized 3-7 days following transfection, and the cells were then transduced with lentiviral vectors encoding the ZFP-TF 8982-KRAB at 10-50 MOI for 3 additional days. See Example 2.

Rat dorsal root ganglia cultures were prepared as described by Burfey et al. (2004) Methods Mol Med 99:189-202. In brief, the ganglia were dissected from Sprague-Dawley rats (150-175 gram, Charles River) and digested with 1.25 mg/ml of collagenase (Sigma) at 37° C. for 2 hours. Following digestion, 1 mg/ml of DNase I was added into the solution and the ganglia were resuspended in DRG growth media (F12 medium (Invitrogen) supplemented with 10% horse serum (Invitrogen), 50 U/ml penicillin, 50 μg/ml streptomycin, 2 mM L-glutamine, 50 μM 5-fluoro-2-deoxyuridine (Sigma), 150 μM uridine (Sigma), and 250 ng/ml nerve growth factor (Invitrogen). Cells were dissociated by mechanical trituration through a fire-polished Pasteur pipette and seeded onto poly-D-Lysine and laminin coated coverslips in 12-well plates. Cells were maintained in DRG growth media at 37° C. with 5% CO₂ and the media were changed every 2-3 days. Rat dorsal root ganglia cultures were transduced with lentiviral or herpes simplex virus vectors encoding 8982-KRAB, GFP, or an unrelated ZFP-TF control at 10-50 MOI for 5-7 days. See Example 3.

### C. Nav1.8 Gene Expression Analysis

Total RNA was isolated using either the High Pure RNA kit (Roche Diagnostics) or the RNeasy kit (Qiagen, Valencia, Calif.) according to the manufacturer's recommendations. Real-time quantitative RT-PCR using Taqman® chemistry in a 96-well format on an ABI 7700 SDS machine (Perkin Elmer) was performed as described previously (see Liu et al. (2001) J Biol Chem).

Human Nav1.8 Taqman® Assay (HsO0197867 ml, Applied Biosystems) and rat Nav1.8 primer/probe set (TCTTCCAGAGAAAGTCGAGTACGTC (SEQ ID NO:51), TAGACAAAACCCTCTTGCCAGTATC (SEQ ID NO:52), and FAM-TCACTGTCATTTACACCTTCGAGGCTCTGATT-TAMRA (SEQ ID NO:53)) were used to measure human and rat Nav1.8 expression levels, respectively.

The Nav1.8 mRNA expression levels were normalized to GAPDH mRNA using the GAPDH primer/probe sets for human (CCATGTTCGTCATGGGTGTGA (SEQ ID NO:54), CATGGACTGTGGTCATGAGT (SEQ ID NO:55), FAM-TCCTGCACCACCAACTGCTTAGCA-TAMRA (SEQ ID NO:56)) and rat (CCCATGTTTGTGATGGGTGTG (SEQ ID NO:57), ATCCTGCACCACCAACTGCTTAGC (SEQ ID NO:58), and FAM-ATCCTGCACCACCAACTGCTTAGC-TAMRA (SEQ ID NO:59)).

Expression levels of rat Nav1.8 in rat dorsal root ganglia culture was also normalized to DRG neuronal marker peripherin using rat peripherin Taqman® Assay (Rn00561807m1, Applied Biosystems).

### D. Immunocytochemistry and Fluorescence Microscopy

Cultured cells were fixed with 4% paraformaldehyde for 10 min. at room temperature. After 2 washes with PBS, cells were permeablized with 0.5% Saponin/PBS for 10 min. and blocked in 3% BSA/0.1% Saponin/PBS for 30 min. For double immunostaining with Nav1.8 and ZFP-TF, cells were exposed to rabbit anti-Nav1.8 antibody (Alomone Lab, Israel) and mouse anti-flag M2 monoclonal antibody (Sigma), followed by Alexa Fluor 488 conjugated goat anti-rabbit IgG and Alexa Fluor 594 conjugated goat anti-mouse IgG (Invitrogen). Cells were mounted on slides with ProLong Antifade reagent (Invitrogen) and visualized under a fluorescence microscope.

### Example 2

### Repression of Nav1.8 Gene Expression in DAOY Cells

A fusion protein (8982-KRAB) comprising a 6-fingered DNA-binding domain designed to recognize a target site in human Nav1.8 and a repression domain was designed as described above in Example 1 and in U.S. Pat. No. 6,607,882. The amino acid sequence corresponding to each finger of the DNA-binding domain is shown in Table 1 with reference to “8982”.

Sequences encoding the 8982-KRAB fusion protein were introduced into human DAOY cells via lentiviral vectors as described above in Example 1. A lentiviral vector encoding GFP was also prepared for use as a control.

Nav1.8 expression was analyzed by real-time RT-PCR with normalization to GAPDH as described above with reference to gene expression analysis.

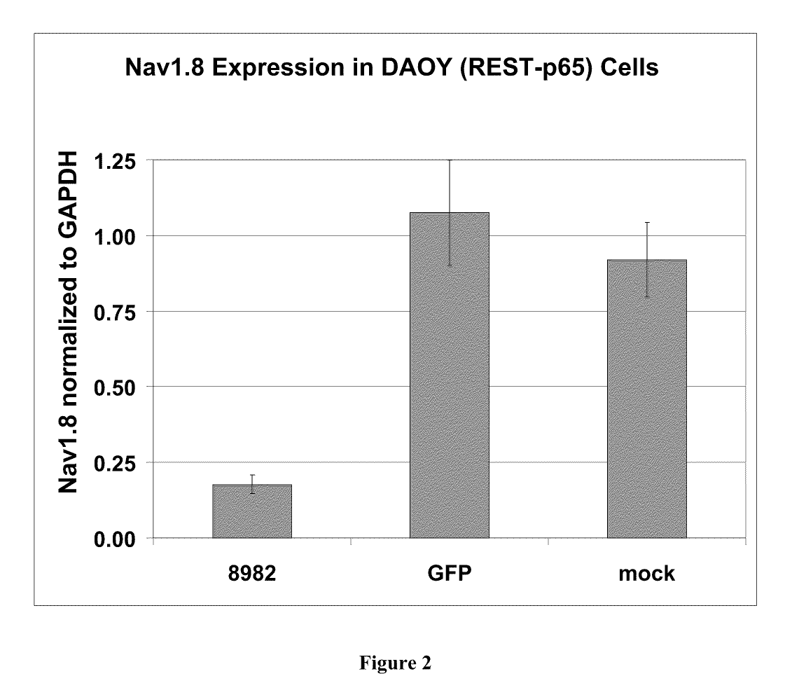

FIG. 2 shows the results of repression of human Nav1.8 expression using 8982-KRAB. Administration of this Nav1.8-targeted ZFP significantly repressed human Nav1.8 expression.

### Example 3

### Repression of Nav1.8 in Rat DRG Neurons

### A. Gene Expression

The activity of ZFP-TF 8982-KRAB was tested in a primary culture of rat dorsal root ganglion neurons transduced with lentiviral or herpes simplex virus (HSV) vectors as described previously. Nav1.8 gene expression was analyzed by real-time RT-PCR as described above, and normalized to either GAPDH mRNA or peripherin mRNA (a specific marker for sensory neurons).

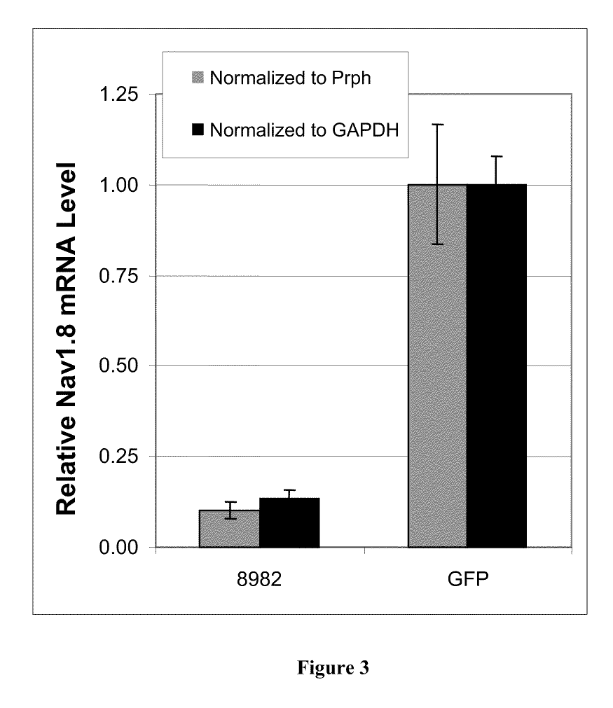

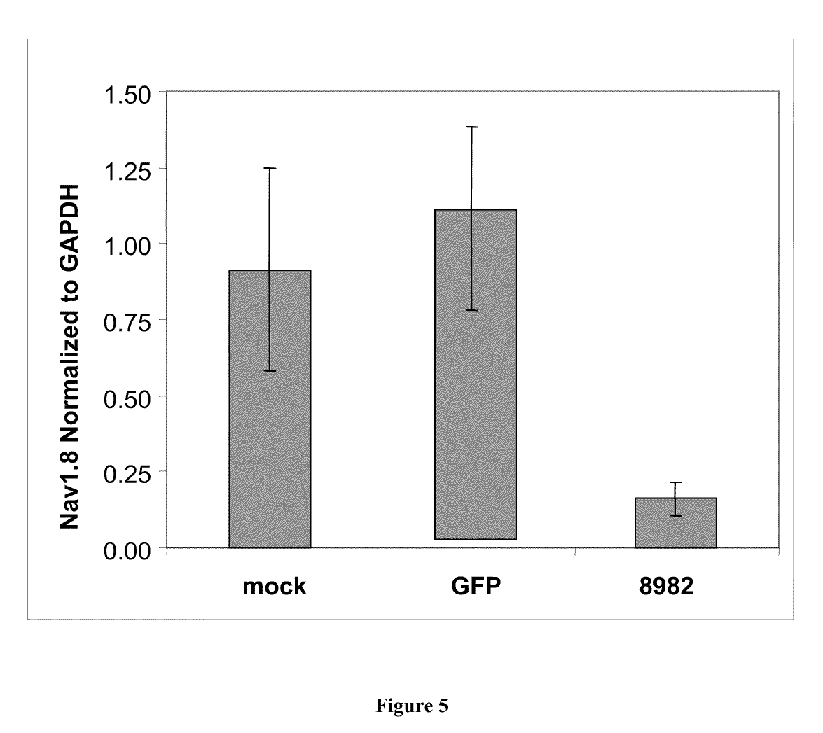

As shown in FIG. 3, 8982-KRAB, transduced via a lentiviral vector, significantly repressed Nav1.8 gene expression, resulting in a ˜10-fold reduction in Nav1.8 mRNA levels, compared to GFP controls. FIG. 5 shows a similar repression of Nav1.8 gene expression when 8982-KRAB was delivered via an HSV vector.

### B. Protein Expression

Repression of Nav1.8 was also demonstrated at the protein level. ZFP-TF 8982-KRAB depressed Nav1.8 protein levels in the rat DRG neurons following transduction via lentiviral vectors as described previously, as compared to an unrelated control ZFP-TF.

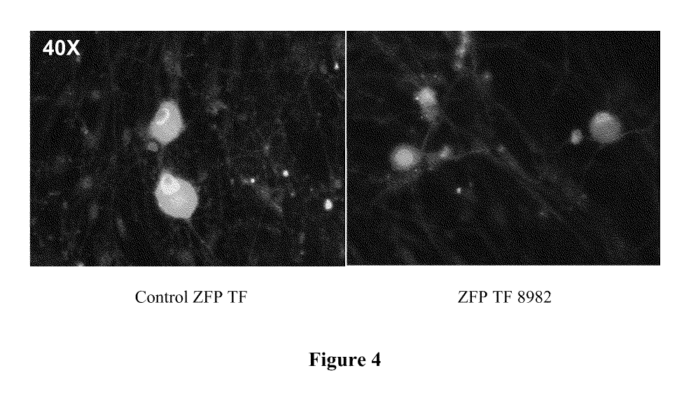

FIG. 4 shows the results of 8982-KRAB on Nav1.8 protein levels in the rat DRG neurons. The antibodies used for visualizing the fluorescence micrographs were as described above in Example 1. The light areas in the micrograph panel labeled “Control ZFP TF” correspond principally to an immunostain of rabbit anti-Nav1.8 antibody bound to an Alexa Fluor 488 conjugated goat anti-rabbit IgG antibody, and illustrate the high level of Nav1.8 protein ordinarily present in the rat DRG sensory neurons. In contrast, the light areas in the micrograph panel labeled “ZFP TF 8982” correspond principally to an immunostain of mouse anti-flag M2 monoclonal antibody bound to an Alexa Fluor 594 conjugated goat anti-mouse IgG antibody, and illustrate the relatively high concentration of ZFP-TF 8982-KRAB present in the cells transduced with the test ZFP. The relative absence of Nav1.8 protein in the rat DRG sensory neurons transduced with 8982-KRAB is apparent in the micrograph.

Thus, Nav1.8-targeted ZFPs repress expression of Nav1.8 at the nucleotide and protein levels.

### Example 4

### Repression of Nav1.8 in a Neuropathic Pain Rat Model

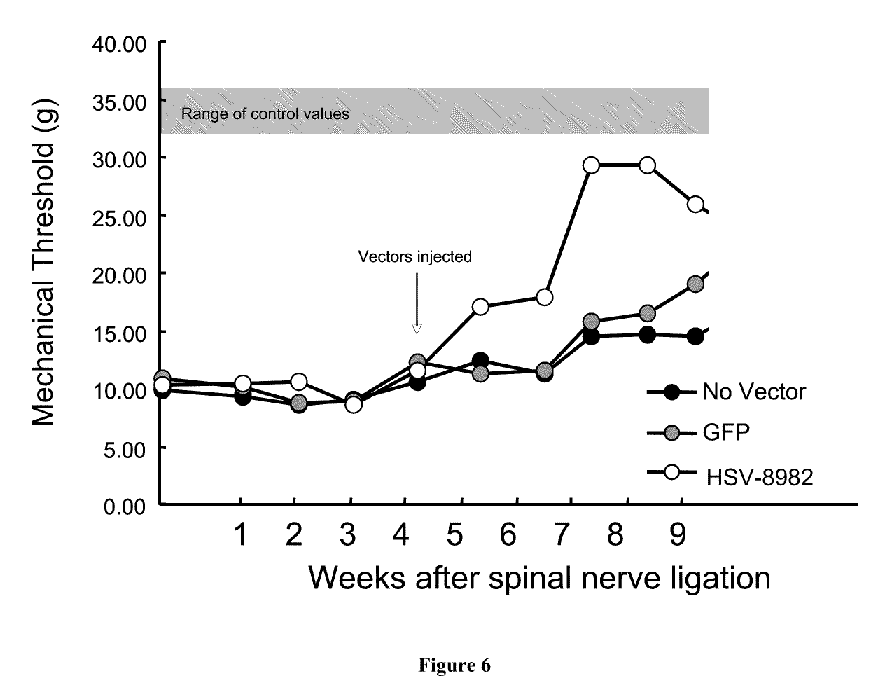

The efficacy of ZFP-TF 8982-KRAB in alleviating neuropathic pain was tested in a rat model of spinal nerve ligation (SNL) in which the rat L5 spinal nerve was tightly ligated and mechanical allodynia develops on the ipsilateral paw. Four weeks following SNL, rats were inoculated into the footpad with a herpes simplex virus (HSV) vector comprising ZFP-TF 8982-KRAB (HSV-8982) or Green Fluorescent Protein (GFP) (5 rats/group), and mechanical allodynia was measured weekly. As shown in FIG. 6, rats that were injected with HSV-8982 showed improved allodynia one week after injection compared to GFP and no-vector controls. At the end of three and four week timepoints, the HSV-8982 treated rats exhibited mechanical thresholds close to that of normal rats, and significantly higher thresholds than those rats treated with GFP or no-vector control animals. This result demonstrates the efficacy of the ZFP repressor of Nav1.8 in an in vivo neuropathic pain model.

### Example 5

### Knock Down of Tetrodotoxin-Resistant (TTX-R) Sodium Current in Rat Neonatal Dorsal Root Ganglia Cells by 8982-KRAB

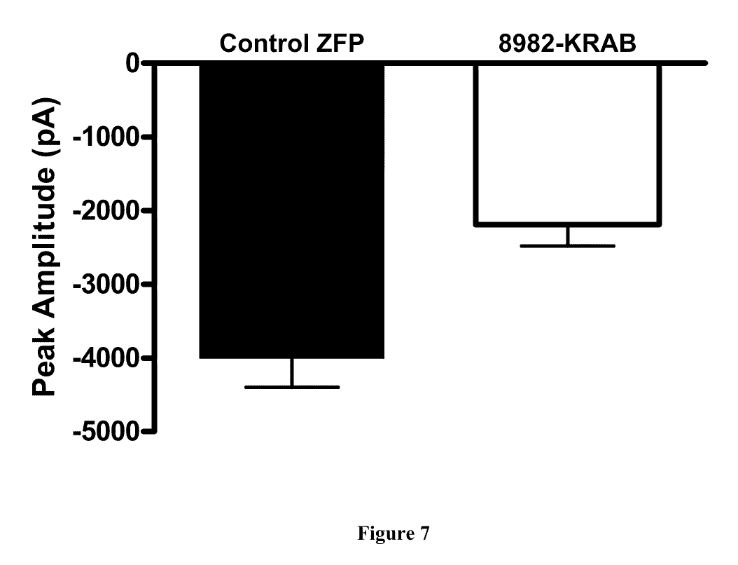

The efficacy of ZFP-TF 8982-KRAB to knock down the function of Nav1.8 was tested by recording sodium currents in cultured rat neonatal dorsal root ganglia (DRG) neurons transduced with lentiviral vectors as described previously. The lentiviral vectors were constructed to co-express both the ZFP-TF (8982-KRAB or control) and GFP so that the transduced cells could be identified by the presence of the green fluorescence under a fluorescence microscope. Forty eight hours following transduction, the TTX-R sodium currents were measured in small and medium-sized transduced DRG neurons by whole-cell patch-clamp recordings, as described by Zhou et al., 2003. J Pharmacol Exp Ther 306:498-504. The currents measured under these conditions represent mostly the Nav1.8-mediated TTX-R sodium currents. As shown in FIG. 7, ZFP-TF 8982-KRAB produced a ˜2-fold reduction of TTX-R currents, compared to a ZFP-TF control. This result demonstrates that ZFP-TF 8982 is capable of blocking the functional Nav1.8 channels.

It is understood that the examples and embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entireties for all purposes to the same extent as if each individual publication, patent or patent application were specifically and individually indicated to be so incorporated by reference.

1. A polynucleotide encoding a protein comprising an engineered zinc finger DNA-binding domain, wherein the DNA-binding domain comprises six zinc fingers denoted F1 through F6 in order from N-terminus to C-terminus and the amino acid sequence of recognition regions F1, F3, F5, and F6 of the zinc fingers is as follows:

| | | |
|---|---|---|
| F1: | RSDVLSQ | (SEQ ID NO:15) |
| F3: | RSDNLSR | (SEQ ID NO:13) |
| F5: | QSGNLAR | (SEQ ID NO:11) |
| F6: | QSGNLAR. | (SEQ ID NO:11) |

2. The polynucleotide of claim 1, wherein the amino acid sequence of recognition regions F2 and F4 is as follows:

| | | |
|---|---|---|
| F2: | RSDNLSV | (SEQ ID NO:14) |
| F4: | TNQNRIT. | (SEQ ID NO:12) |

3. The polynucleotide of claim 1, wherein the amino acid sequence of recognition region F2 is selected from the group consisting of YSRGLWA (SEQ ID NO:16), WPGSLSN (SEQ ID NO:17), WRPNLVA (SEQ ID NO:18), APRYLWQ (SEQ ID NO:19), LLKYLAT (SEQ ID NO:20), SSRYLWQ (SEQ ID NO:23), HPRYLWQ (SEQ ID NO:24), QRRYLWA (SEQ ID NO:26), and QKRYLWQ (SEQ ID NO:28), and the amino acid sequence of recognition region F4 is selected from the group consisting of TNQNRIT (SEQ ID NO:12), LKRTLMV (SEQ ID NO:21), LLQTLSS (SEQ ID NO:22), LHRTLTV (SEQ ID NO:25), VRCNLTK (SEQ ID NO:27), LRRTLHM (SEQ ID NO:29), and LKNALR1 (SEQ ID NO:30).

4-11. (canceled)

12. The polynucleotide of claim 1, further comprising a transcriptional repression domain.

13. The polynucleotide of claim 12, wherein the transcriptional repression domain is a KRAB domain.

14. The polynucleotide of claim 1, further comprising a transcriptional activation domain.

15. A polypeptide encoded by the polynucleotide of claim 1.

16. The polypeptide of claim 15, further comprising a transcriptional repression domain.

17. The polypeptide of claim 16, wherein the transcriptional repression domain is a KRAB domain.

18. The polypeptide of claim 15, further comprising a transcriptional activation domain.

19. The zinc finger protein of claim 22 selected from any one of the proteins provided in Table 1, or a nucleic acid encoding the protein.

20-21. (canceled)

22. A zinc finger protein comprising a zinc finger comprising an amino sequence shown in Table 1, or a nucleic acid encoding the protein.

23. (canceled)

24. A method for treating chronic pain in a subject, the method comprising:
introducing a nucleic acid into a subject, wherein the nucleic acid encodes a polypeptide, the polypeptide comprising:
(i) a zinc finger DNA-binding domain that is engineered to bind to a target site in Nav10.8, the zinc finger DNA-binding domain comprising six zinc fingers denoted F1 through F6 in order from N-terminus to C-terminus and wherein the amino acid sequence of recognition regions F1, F3, F5, and F6 of the zinc fingers is as follows:

| | | |
|---|---|---|
| F1: | RSDVLSQ | (SEQ ID NO:15) |
| F3: | RSDNLSR | (SEQ ID NO:13) |
| F5: | QSGNLAR | (SEQ ID NO:11) |
| F6: | QSGNLAR; | (SEQ ID NO:11) |

and
(ii) a transcriptional repression domain;
such that the nucleic acid is expressed in one or more cells of the subject, whereby the polypeptide binds to the target site and represses transcription of the Nav1.8 gene.

25. The method of claim 24, wherein the target site in the Nav1.8 gene comprises a nucleic acid sequence of 5′-GAAGAAgAATGAGAAGATG (SEQ ID NO:2).

26. The method of claim 24, wherein the transcriptional repression domain is a KRAB domain.

27. The method of claim 24, wherein the amino acid sequence of recognition regions F2 and F4 is as follows:

| | | |
|---|---|---|
| F2: | RSDNLSV | (SEQ ID NO:14) |
| F4: | TNQNRIT. | (SEQ ID NO:12) |

28. The method of claim 24, wherein the amino acid sequence of recognition region F2 is selected from the group consisting of YSRGLWA (SEQ ID NO:16), WPGSLSN (SEQ ID NO:17) WRPNLVA (SEQ ID NO:18), APRYLWQ (SEQ ID NO:19), LLKYLAT (SEQ ID NO:20) SSRYLWO (SEQ ID NO:23), HPRYLWQ (SEQ ID NO:24), QRRYLWA (SEQ ID NO:26), and QKRYLWQ (SEQ ID NO:28), and the amino acid sequence of recognition region F4 is selected from the group consisting of TNQNRIT (SEQ ID NO:12), LKRTLMV (SEQ ID NO:21), LLQTLSS (SEQ ID NO:22), LHRTLTV (SEQ ID NO:25), VRCNLTK (SEQ ID NO:27), LRRTLHM (SEQ ID NO:29), and LKNALR1 (SEQ ID NO:30).

29-69. (canceled)

70. The method of claim 24, wherein the chronic pain comprises neuropathic pain.

71. (canceled)


SEQUENCE LISTING (60 sequences)

SEQ ID NO: 1 - protein, 25 aa, Artificial Sequence
  Description of Artificial Sequence Synthetic peptide
CXXXXCXXXXXXXXXXXXHXXXXXH

SEQ ID NO: 2 - DNA, 19 bp, Homo sapiens
gaagaagaatgagaagatg

SEQ ID NO: 3 - DNA, 19 bp, Rattus sp.
caagaagaatgagaagatg

SEQ ID NO: 4 - DNA, 3 bp, Artificial Sequence
  Description of Artificial Sequence Synthetic oligonucleotide
gaa

SEQ ID NO: 5 - DNA, 3 bp, Artificial Sequence
  Description of Artificial Sequence Synthetic oligonucleotide
caa

SEQ ID NO: 6 - DNA, 4 bp, Artificial Sequence
  Description of Artificial Sequence Synthetic oligonucleotide
gaag

SEQ ID NO: 7 - DNA, 3 bp, Artificial Sequence
  Description of Artificial Sequence Synthetic oligonucleotide
aat

SEQ ID NO: 8 - DNA, 3 bp, Artificial Sequence
  Description of Artificial Sequence Synthetic oligonucleotide
gag

SEQ ID NO: 9 - DNA, 3 bp, Artificial Sequence
  Description of Artificial Sequence Synthetic oligonucleotide
aag

SEQ ID NO: 10 - DNA, 3 bp, Artificial Sequence
  Description of Artificial Sequence Synthetic oligonucleotide
atg

SEQ ID NO: 11 - protein, 7 aa, Artificial Sequence
  Description of Artificial Sequence Synthetic peptide
QSGNLAR

SEQ ID NO: 12 - protein, 7 aa, Artificial Sequence
  Description of Artificial Sequence Synthetic peptide
TNQNRIT

SEQ ID NO: 13 - protein, 7 aa, Artificial Sequence
  Description of Artificial Sequence Synthetic peptide
RSDNLSR

SEQ ID NO: 14 - protein, 7 aa, Artificial Sequence
  Description of Artificial Sequence Synthetic peptide
RSDNLSV

SEQ ID NO: 15 - protein, 7 aa, Artificial Sequence
  Description of Artificial Sequence Synthetic peptide
RSDVLSQ

SEQ ID NO: 16 - protein, 7 aa, Artificial Sequence
  Description of Artificial Sequence Synthetic peptide
YSRGLWA

SEQ ID NO: 17 - protein, 7 aa, Artificial Sequence
  Description of Artificial Sequence Synthetic peptide
WPGSLSN

SEQ ID NO: 18 - protein, 7 aa, Artificial Sequence
  Description of Artificial Sequence Synthetic peptide
WRPNLVA

SEQ ID NO: 19 - protein, 7 aa, Artificial Sequence
  Description of Artificial Sequence Synthetic peptide
APRYLWQ

SEQ ID NO: 20 - protein, 7 aa, Artificial Sequence
  Description of Artificial Sequence Synthetic peptide
LLKYLAT

SEQ ID NO: 21 - protein, 7 aa, Artificial Sequence
  Description of Artificial Sequence Synthetic peptide
LKRTLMV

SEQ ID NO: 22 - protein, 7 aa, Artificial Sequence
  Description of Artificial Sequence Synthetic peptide
LLQTLSS

SEQ ID NO: 23 - protein, 7 aa, Artificial Sequence
  Description of Artificial Sequence Synthetic peptide
SSRYLWQ

SEQ ID NO: 24 - protein, 7 aa, Artificial Sequence
  Description of Artificial Sequence Synthetic peptide
HPRYLWQ

SEQ ID NO: 25 - protein, 7 aa, Artificial Sequence
  Description of Artificial Sequence Synthetic peptide
LHRTLTV

SEQ ID NO: 26 - protein, 7 aa, Artificial Sequence
  Description of Artificial Sequence Synthetic peptide
QRRYLWA

SEQ ID NO: 27 - protein, 7 aa, Artificial Sequence
  Description of Artificial Sequence Synthetic peptide
VRCNLTK

SEQ ID NO: 28 - protein, 7 aa, Artificial Sequence
  Description of Artificial Sequence Synthetic peptide
QKRYLWQ

SEQ ID NO: 29 - protein, 7 aa, Artificial Sequence
  Description of Artificial Sequence Synthetic peptide
LRRTLHM

SEQ ID NO: 30 - protein, 7 aa, Artificial Sequence
  Description of Artificial Sequence Synthetic peptide
LKNALRI

SEQ ID NO: 31 - protein, 5 aa, Artificial Sequence
  Description of Artificial Sequence Synthetic peptide
TGEKP

SEQ ID NO: 32 - protein, 5 aa, Artificial Sequence
  Description of Artificial Sequence Synthetic peptide
GGGGS

SEQ ID NO: 33 - protein, 8 aa, Artificial Sequence
  Description of Artificial Sequence Synthetic peptide
GGRRGGGS

SEQ ID NO: 34 - protein, 9 aa, Artificial Sequence
  Description of Artificial Sequence Synthetic peptide
LRQRDGERP

SEQ ID NO: 35 - protein, 12 aa, Artificial Sequence
  Description of Artificial Sequence Synthetic peptide
LRQKDGGGSERP

SEQ ID NO: 36 - protein, 16 aa, Artificial Sequence
  Description of Artificial Sequence Synthetic peptide
LRQKDGGGSGGGSERP

SEQ ID NO: 37 - protein, 25 aa, Artificial Sequence
  Description of Artificial Sequence Synthetic peptide
CXXXXCXXXXXXXXXXXXHXXXXXH

SEQ ID NO: 38 - protein, 30 aa, Mus sp.
YACPVESCDRRFSRSDELTRHIRIHTGQKP

SEQ ID NO: 39 - protein, 28 aa, Mus sp.
FQCRICMRNFSRSDHLTTHIRTHTGEKP

SEQ ID NO: 40 - protein, 27 aa, Mus sp.
FACDICGRKFARSDERKRHTKIHLRQK

SEQ ID NO: 41 - DNA, 9 bp, Mus sp.
gcgtgggcg

SEQ ID NO: 42 - DNA, 9 bp, Artificial Sequence
  Description of Artificial Sequence Synthetic oligonucleotide
ggggcgggg

SEQ ID NO: 43 - protein, 7 aa, Simian virus 40
PKKKRKV

SEQ ID NO: 44 - protein, 8 aa, Artificial Sequence
  Description of Artificial Sequence Synthetic peptide
DYKDDDDK

SEQ ID NO: 45 - DNA, 20 bp, Artificial Sequence
  Description of Artificial Sequence Synthetic oligonucleotide
ccaacgaagacaagatctgc

SEQ ID NO: 46 - DNA, 18 bp, Artificial Sequence
  Description of Artificial Sequence Synthetic oligonucleotide
tcctgcgtcgagagagct

SEQ ID NO: 47 - DNA, 23 bp, Artificial Sequence
  Description of Artificial Sequence Synthetic oligonucleotide
cgcccgaacagggacctgaaagc

SEQ ID NO: 48 - DNA, 25 bp, Artificial Sequence
  Description of Artificial Sequence Synthetic oligonucleotide
tgaaacatacgttcccaaagagttt

SEQ ID NO: 49 - DNA, 26 bp, Artificial Sequence
  Description of Artificial Sequence Synthetic oligonucleotide
ctctccttctcagaaagtgtgcatat

SEQ ID NO: 50 - DNA, 27 bp, Artificial Sequence
  Description of Artificial Sequence Synthetic oligonucleotide
tgctgaaacattcaccttccatgcaga

SEQ ID NO: 51 - DNA, 25 bp, Artificial Sequence
  Description of Artificial Sequence Synthetic oligonucleotide
tcttccagagaaagtcgagtacgtc

SEQ ID NO: 52 - DNA, 25 bp, Artificial Sequence
  Description of Artificial Sequence Synthetic oligonucleotide
tagacaaaaccctcttgccagtatc

SEQ ID NO: 53 - DNA, 32 bp, Artificial Sequence
  Description of Artificial Sequence Synthetic oligonucleotide
tcactgtcatttacaccttcgaggctctgatt

SEQ ID NO: 54 - DNA, 21 bp, Artificial Sequence
  Description of Artificial Sequence Synthetic oligonucleotide
ccatgttcgtcatgggtgtga

SEQ ID NO: 55 - DNA, 20 bp, Artificial Sequence
  Description of Artificial Sequence Synthetic oligonucleotide
catggactgtggtcatgagt

SEQ ID NO: 56 - DNA, 24 bp, Artificial Sequence
  Description of Artificial Sequence Synthetic oligonucleotide
tcctgcaccaccaactgcttagca

SEQ ID NO: 57 - DNA, 21 bp, Artificial Sequence
  Description of Artificial Sequence Synthetic oligonucleotide
cccatgtttgtgatgggtgtg

SEQ ID NO: 58 - DNA, 24 bp, Artificial Sequence
  Description of Artificial Sequence Synthetic oligonucleotide
atcctgcaccaccaactgcttagc

SEQ ID NO: 59 - DNA, 24 bp, Artificial Sequence
  Description of Artificial Sequence Synthetic oligonucleotide
atcctgcaccaccaactgcttagc

SEQ ID NO: 60 - DNA, 13 bp, Homo sapiens
gaagaatgagaag